(19) United States
(12) Patent Application Publication
(10) Pub. No.: US 2022/0409730 A1
(43) Pub. Date: Dec. 29, 2022

(54) DOSAGE FORM FOR EXTENDED RELEASE OF AN ANTIBODY OR LARGE PROTEIN

(71) Applicant: InnoCore Technologies Holding B.V., Groningen (NL)

(72) Inventors: Rob STEENDAM, Groningen (NL); Maria COIMBRA, Haarlem (NL); Joana Catarina RIBEIRO ARAUJO, Soesterberg (NL); Johan ZUIDEMA, Aduard (NL); Tanja HENZLER, Mannheim (DE); Alexandra KROG, Bodenheim (DE)

(73) Assignee: InnoCore Technologies Holding B.V., Groningen (NL)

(21) Appl. No.: 17/773,520

(22) PCT Filed: Oct. 30, 2020
(86) PCT No.: PCT/NL2020/050674

§ 371 Date: Apr. 29, 2022

(30) Foreign Application Priority Data
Nov. 1, 2019 (EP) 19206786.6

(51) Int. Cl. A61K 47/34; A61K 47/68; A61K 9/00; A61K 9/16

(52) U.S. Cl. CPC A61K 47/34; A61K 47/6835; A61K 9/0019; A61K 9/1647

(57) ABSTRACT

The present disclosure relates to dosage forms for extended release of an antibody or large protein. The extended release dosage forms comprise a biodegradable multi-block copolymer matrix and provide for extended release of antibody or large protein over a desired time period.

### I. BACKGROUND

Antibodies and antigen binding fragments, such as monoclonal antibodies, bispecific antibodies, tri-specific antibodies, antigen binding fragments (Fab), antibody drug conjugates (ADCs) and other high molecular weight biologicals such as Fc-fusion proteins, enzymes, growth factors and coagulation factors are of growing importance in the development of more specific medicines for more effective pharmacotherapies with fewer side effects. The rapid advances in this field along with the large-scale production of these compounds by recombinant DNA technology—among other techniques—have induced a huge increase in the number of antibodies, antigen binding fragments and other large protein-derived medicine in development and already reaching the market. Unfortunately, the development of antibodies, antigen binding fragments and other large therapeutic proteins have far outpaced the ability to deliver these compounds systemically or locally using convenient and effective delivery systems.

Biodegradable polymers have received increased attention over the past decade for use in long-acting parenteral controlled release systems, either for systemic or site-specific drug delivery. Biodegradable controlled release formulations can significantly improve the pharmacokinetics of therapeutic compounds. This is especially relevant in the treatment of chronic diseases and for compounds with a narrow therapeutic window since systemic plasma concentrations can be reduced with concurrent reduction in undesirable side effects. Additionally, many new biologically active compounds have short half-lives, necessitating frequent injection to achieve therapeutically effective plasma levels. Patient compliance and the high costs associated with frequent dosing regimens for parenterally administered biologically active compounds have increased the interest in biodegradable parenteral sustained release dosage forms.

Poly(D,L-lactic acid) (PLA) and copolymers of lactic acid and glycolic acid, also known as PLGA copolymers, are the most widely applied biodegradable polymers for use in parenteral sustained release depot formulations. PLGA and PLA (together called (PL(G)A) copolymers have been successfully used for the development of sustained release depot formulations for small molecules, such as risperidone, and therapeutic peptides such as leuprolide, goserelin or octreotide.

PL(G)A polymers have, however, several drawbacks that limit their use and make them unsuitable for the delivery of protein therapeutics. Firstly, PL(G)A copolymers are relatively hydrophobic polymers and do not provide an optimal environment for encapsulated proteins. Proteins may adsorb to the polymer, resulting in slow and incomplete release, protein unfolding and/or aggregation. Secondly, the ability to manipulate the release of large protein molecules is limited since diffusion of such compounds through the relatively rigid and non-swellable PL(G)A matrices is negligible. The release of proteins from PL(G)A copolymers therefore depends on diffusion via pores present in the matrix and on the degradation of the matrix. Typically, the encapsulated protein remains entrapped in the polymer matrix until the moment the latter has degraded to such an extent that it loses its integrity or dissolves, resulting in biphasic or triphasic degradation-dependent release profiles typically obtained for PL(G)A-based depot formulations. Finally, during degradation of PL(G)A copolymers, acidic moieties are formed that accumulate in the rigid and non-swellable PL(G)A matrix resulting in the formation of an acidic micro-environment in the polymer matrix with in situ pHs that can be as low as 1-2. Under such acidic conditions encapsulated proteins may form aggregates leading to incomplete protein release. Moreover, the low pH may have a deleterious effect on the structural integrity and biological activity of the encapsulated peptide or protein, potentially leading to reduced therapeutic efficacy and enhanced immunogenicity. Chemical modification of proteins and peptides, such as acylation and adduct formation have been reported for PL(G)A based sustained release formulations.

Biodegradable phase separated segmented multi-block copolymers (SynBiosys®, InnoCore Technologies B.V, Groningen, The Netherlands) as disclosed in WO-A-2012/005594 and WO-A-2013/015685 have been developed to deliver peptides and proteins structurally intact and biologically active over extended periods of time up to three to six months (Stankovic et al., Eur. J. Pharm. Sci. 2013, 49(4), 578-587; Teekamp et al. Int. J. Pharm. 2017, 534(1-2), 229-236; Teekamp et al., J. Controlled Release 2018, 269(10), 258-265; Scheiner et al., ACS Omega 2019, 4(7), 11481-11492). SynBiosys multi-block copolymers are typically composed of two different blocks in which commonly used monomers including D,L-lactide, L-lactide, glycolide, ε-caprolactone, p-dioxanone and/or poly(ethylene glycol) (PEG) are copolymerized into a low molecular weight polymers (pre-polymers), which are linked together with a diisocyanate, typically 1,4-butanediisocyanate. By using two chemically and physically distinct pre-polymer blocks, such as a hydrophilic amorphous and a hydrophobic crystalline block, a phase separated segmented multi-block copolymer is obtained that provides mechanisms for long term release of drugs including peptides and proteins. The hydrophilic amorphous blocks typically contain a high content of poly(ethylene glycol) (PEG) which leads to swelling of the multi-block copolymer under aqueous conditions. The hydrophobic crystalline blocks act as physical crosslinks. Examples of peptides and proteins that were successfully encapsulated in and released structurally intact from SynBiosys-based formulations include among others goserelin (1269 g/mol), exenatide (4187 g/mol), recombinant insulin (5.8 kDa), insulin-like growth factor (7.6 kDa) lysozyme (14.7 kDa), carbonic anhydrase (29 kDa), bovine serum albumin (66.5 kDa) and hepatocyte growth factor (69 kDa).

However, antibodies and antigen binding fragments, monoclonal antibodies, bispecific antibodies, tri-specific antibodies, antigen binding fragments (Fab), antibody drug conjugates (ADCs) and other high molecular weight biologicals such as Fc-fusion proteins may have molecular weights up to or even exceeding 200 kDa and have complex three-dimensional structures.

Unfortunately, the delivery of monoclonal antibodies is burdensome. Monoclonal antibodies are typically administered by intravenous (IV) infusion at low concentrations, which can take multiple hours to deliver the complete dose, cause patient discomfort, and increase the risk of infection. Furthermore, the patient needs to come to the hospital frequently to undergo the infusion. For example, Bevacizumab (Avastin®, Genentech, Inc.), a humanised anti-VEGF monoclonal IgG₁ antibody with a molecular weight of 149 kDa which is used in the treatment of various cancers, is administered as a 25 mg/ml solution by infusion for up to 90 minutes every 2 weeks.

There is a long-felt desire to replace this inconvenient and burdensome administration procedure by a more patient-friendly subcutaneous administration. The use of highly concentrated (and viscous) antibody solutions has enabled the subcutaneous delivery of high doses of antibodies and antigen binding fragments, but has not solved the need for frequent administration. There is still a need in the art for extended release dosage forms that can be administered subcutaneously and provide extended release of structurally intact and biologically active antibodies and antigen binding fragments. The present invention satisfies this need.

### II. SUMMARY

The present disclosure relates to the long felt need in the art for parenteral extended release formulations for large proteins and antibodies.

Accordingly, in an aspect the invention is directed to a dosage form for extended release of an antibody (or an antigen binding fragment thereof), comprising

(a) an antibody (or an antigen binding fragment thereof);
(b) a biodegradable multi block copolymer matrix,
wherein the antibody (or the antigen binding fragment thereof) is present in the multi block copolymer matrix,
wherein the biodegradable multi-block copolymer comprises one or more biodegradable, phase separated, thermoplastic multi-block copolymers comprising at least one amorphous hydrolysable pre-polymer (A) segment and at least one semi-crystalline hydrolysable pre-polymer (B) segment,
wherein
said multi-block copolymer under physiological conditions has a T_{g} of about 37° C. or less and a Tₘ of about 50° C. to about 110° C.;
the segments are linked by a multifunctional chain extender;
the segments are randomly distributed over the polymer chain; and
the pre-polymer (B) segment comprises a X—Y—X triblock copolymer wherein Y is a polymerisation initiator, and X is a poly(p-dioxanone) segment with a block length expressed in p-dioxanone monomer units of about 7 or more.

In a further aspect the invention is directed to a dosage form for extended release of a protein of about 70 kDa or more, comprising

(a) a protein of about 70 kDa or more;
(b) a biodegradable multi block copolymer matrix,
wherein the protein is present in the multi-block copolymer matrix,
wherein the biodegradable multi-block copolymer comprises one or more biodegradable, phase separated, thermoplastic multi-block copolymers comprising at least one amorphous hydrolysable pre-polymer (A) segment and at least one semi-crystalline hydrolysable pre-polymer (B) segment, wherein
said multi-block copolymer under physiological conditions has a T_{g} of about 37° C. or less and a Tₘ of about 50° C. to about 110° C.;
the segments are linked by a multifunctional chain extender;
the segments are randomly distributed over the polymer chain; and
the pre-polymer (B) segment comprises a X—Y—X triblock copolymer wherein Y is a polymerisation initiator, and X is a poly(p-dioxanone) segment with a block length expressed in p-dioxanone monomer units of about 7 or more.

In some embodiments, the dosage forms exhibit release profiles of between about 1 week and about 2 months. In other embodiments, the dosage forms described herein can have extended release profiles between about 1 week and about 6 months.

The multi-block copolymer may comprise, or alternatively consists essentially of, poly(ethylene glycol) (PEG) (or a PEG-containing polymeric block), and one or more other polymeric blocks. The polymer matrix may be in the form of a microsphere or a microparticle, but may also be comprised in another application form, such as a nanosphere or nanoparticle, a rod, an implant, a coating, a film, a sheet, a tube, a membrane, a mesh, fibres, a plug, a scaffold or (an in situ forming) gel.

Antibody and large protein extended release formulations described herein may be useful in a variety of treatments, including but not limited to treatment of cancer, neurodegenerative disease, autoimmune disease, cardiovascular diseases, transplant rejections, inflammatory diseases, viral infections. Thus, further aspects of the disclosure relate to methods of administering the extended release antibody formulations described herein, as well as methods of treatment employing the extended release antibody formulations described herein. Such methods include a variety of treatments, including but not limited to treatments for cancer, neurodegenerative diseases, autoimmune diseases, cardiovascular diseases, transplant rejections, inflammatory diseases and viral infections.

In another embodiment, encompassed are methods of administering the described extended release antibody dosage forms according to a regimen that achieves or approximates in vivo plasma levels within the therapeutic window of the antibody.

The inventions described and claimed herein have many attributes and embodiments including, but not limited to, those set forth or described or referenced in this Summary. It is not intended to be all-inclusive and the inventions described and claimed herein are not limited to or by the features or embodiments identified in this Summary, which is included for purposes of illustration only and not restriction. Additional embodiments may be disclosed in the Description of the Figures and Detailed Description below.

### III. BRIEF DESCRIPTION OF THE DRAWINGS

FIG. 1. Scanning electron microscopy images of mAbX-loaded microspheres with 5% mAbX target loading. The microspheres were composed of blends of 50CP30C40-LL40 and 50CP10C20-LL40 (100:0, 50:50, 25:75 and 0:100 w/w) and prepared via the W/O/O-based microencapsulation process.

FIG. 2. Scanning electron microscopy images of mAbX-loaded microspheres with 5% mAbX target loading. The microspheres were composed of blends of 50CP30C40-LL40 and 50CP10C20-LL40 (100:0, 50:50, 25:75 and 0:100 w/w) and were prepared via the W/O/W-based microencapsulation process.

FIG. 3. Cumulative release of mAbX from mAbX-loaded microspheres with 5% mAbX target loading. The microspheres were composed of blends of 50CP30C40-LL40 and 50CP10C20-LL40 (100:0, 50:50. 25:75 and 0:100 w/w) and were prepared via the W/O/O-based microencapsulation process.

FIG. 4. Cumulative release of mAbX from mAbX-loaded microspheres with 5% mAbX target loading. The microspheres were composed of blends of 50CP30C40-LL40 and 50CP10C20-LL40 (100:0, 50:50, 25:75 and 0:100 w/w) and prepared via the W/O/W-based microencapsulation process.

FIG. 5. Cumulative release of mAbX from mAbX-loaded microspheres with 10% mAbX target loading. The microspheres were composed of blends of 50CP30C40-LL40 and 50CP10C20-LL40 (100:0, 90:10 and 80:20 w/w) and prepared via the W/O/W-based microencapsulation process.

FIG. 6. Cumulative release of mAbX from mAbX-loaded microspheres with ˜15% mAbX target loading. The microspheres were composed of 80:20 (AMD17042) and 90:10 w/w (AMD17038) blends of 50CP30C40-LL40 and 50CP10C20-LL40 and prepared via the W/O/W-based microencapsulation process using a 15 wt. % polymer solution concentration.

FIG. 7. Scanning electron microscopy images of mAbX-loaded microspheres with 9.3% (AMD17022) and 19% (AMD17023) mAbX loading. The microspheres were composed of 90:10 w/w blends of 50CP30C40-LL40 and 50CP10C20-LL40 and prepared via the W/O/O-based microencapsulation process.

FIG. 8. Cumulative release of mAbX from mAbX-loaded microspheres with 9.3% (AMD17022) and 19% (AMD17023) mAbX loading. The microspheres were composed of 90:10 w/w blends of 50CP30C40-LL40 and 50CP10C20-LL40 and prepared via the W/O/O-based microencapsulation process.

FIG. 9. Fluorescence spectroscopy (FLS) analysis of in vitro released mAbX showing emission spectra of in vitro release mAbX samples collected at different time points (A), emission maxima of in vitro release mAbX samples collected at different time points relative to native mAbX (B) and formula used to calculate the fraction correctly folded mAbX (C).

FIG. 10. Circular Dichroism (CD) analysis of in vitro released mAbX showing molar ellipticity as a function of wavelength of in vitro release mAbX samples collected at different time points (A), molar ellipticity at the signal peak at 218 nm of in vitro release mAbX samples collected at different time points relative to native mAbX and completely unfolded mAbX (B) and formula used to calculate the fraction correctly folded mAbX (C).

FIG. 11. In vitro release kinetics of mAbX microspheres showing concentration of total and intact mAbX of in vitro release samples collected at different time points as measured by SEC-UPLC and ELISA, and mAbX fraction folded as measured by Fluorescence spectroscopy and Circular Dichroism.

FIG. 12. In vivo pharmacokinetics of mAbX following subcutaneous administration of mAbX MSP (1, 4, 8 mg mAbX) in female NMRI mice (PK study I). Blood samples were collected at indicated time points and mAbX serum concentrations were measured by ELISA.

FIG. 13. In vivo pharmacokinetics of mAbX following subcutaneous administration of mAbX loaded microspheres (1, 2, 4, 8 mg mAbX) in female A431 xenograft-bearing NMRI nude mice (PK study II). Blood samples were collected at indicated time points and mAbX serum concentrations were measured by ELISA.

FIG. 14. Subcutaneous tumour growth as a function of time ((a), and (b) zoom) following subcutaneous administration of mAbX loaded microspheres (1, 2, 4, 8 mg mAbX) in female A431 xenograft-bearing NMRI nude mice. Tumour volume was determined by calliper measurement twice weekly.

FIG. 15. Cumulative release of mAb02 from mAb02-loaded microspheres with 19% mAb02 target loading. The microspheres were composed of blends of 50CP30C40-LL40 and 50CP10C20-LL40 (100:0, 90:10, 80:20, 70:30, 50:50, 25:75 and 100:0 w/w) and prepared via the W/O/O-based microencapsulation process.

FIG. 16. Fraction correctly folded mAb2 during in vitro release of mAb2 from mAb2 loaded microspheres as measured by Fluorescence spectroscopy. A-F represent mAb2 loaded microspheres composed of different polymer compositions.

FIG. 17. In vitro erosion of polymer-only microspheres composed of representative [poly(ε-caprolactone)-PEG-poly(ε-caprolactone)]-b-[poly(L-lactide)] (50CP10C20-LL40) and [poly(ε-caprolactone)-PEG-poly(ε-caprolactone)]-b-[poly(p-dioxanone)] (50CP10C20-D25, 50CP15C20-D25 and 20CP30C40-D23) expressed as remaining mass (%) as a function of time.

FIG. 18. Cumulative release of mAb02 from mAb02-loaded microspheres with 20% mAb02 target loading. The microspheres were composed of blends of 50CP30C40-D25 and 50CP10C20-D25 with blend ratios of 100:0, 90:10, 80:20, 70:30, 50:50, 25:75 and 0:100 w/w) and prepared via the W/O/O-based microencapsulation process.

FIG. 19. In vitro release of mAb02 from mAb02 microspheres composed of blends of 50CP30C40-D25 and 50CP10C20-D25 showing concentration of total and intact mAb02 (as measured by SEC-UPLC) and fraction correctly folded mAb02 (as measured by fluorescence spectroscopy) of in vitro release samples collected at different time points. A-G represent mAb2 loaded microspheres composed of different polymer compositions.

### IV. DETAILED DESCRIPTION

The present invention is directed to extended release dosage forms for large therapeutic proteins or antibodies. There has been a long felt need in the art for such dosage forms as the current dosage forms for large therapeutic proteins or antibodies are solutions for intravenous infusion or intramuscular or subcutaneous injection. Further, the currently available antibodies and therapeutic proteins are typically administered as an aqueous solution. Depending on the half-life of the therapeutic protein or antibody serum levels drop relatively fast following administration due to which the patients need to undergo frequent infusions or receive frequent injections to maintain the desired plasma levels of therapeutic protein or antibody during the treatment period. Frequent infusion or injection can be inconvenient and therefore result in poor patient compliance or lead patients and providers to choose less effective treatments.

The extended release dosage form comprises an antibody (e.g. a monoclonal antibody (mAb), a bispecific antibody, or a tri-specific antibody), an antigen binding fragment (Fab) thereof, an antibody drug conjugate (ADC) or a high molecular weight protein such as a Fc-fusion protein, an enzyme, a growth factor or a coagulation factor encapsulated in a multi-block copolymer matrix. The multi-block copolymer may comprise. or alternatively consists essentially of, poly(ethylene glycol) (PEG) (or a PEG-containing polymeric block), and one or more other polymers.

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

The term “phase separated” as used herein is meant to refer to a system, in particular a copolymer, built of two or more different pre-polymers, of which at least two are (partially) incompatible with each other at body temperature or below (under physiological conditions such as in the human body). Thus, the pre-polymers do not form a homogeneous mixture when combined, neither when combined as a physical mixture of the pre-polymers, nor when the pre-polymers are combined in a single chemical species as “chemical mixture”, viz. as copolymer.

The term “pre-polymer” as used herein is meant to refer to the polymer segments that are randomly linked by a multifunctional chain extender, together making up the multi-block copolymer. Each pre-polymer may be obtained by polymerisation of suitable monomers, which monomers thus are the chemical units of each pre-polymer. The desired properties of the pre-polymers and, by consequence, of the multi-block copolymer, can be controlled by choosing a pre-polymer of a suitable composition and molecular weight (in particular number average molecular weight Mₙ), such that the required Tₘ or T_{g} is obtained.

The term “multi-block” as used herein is meant to refer to the presence of at least two distinct pre-polymer segments in a polymer chain. Additional pre-polymer segments may optionally be present.

The terms “block” and “segment” as used herein are meant to refer to distinct regions in a multi-block copolymer. These terms are used interchangeably herein.

The term “thermoplastic” as used herein is meant to refer to the non-crosslinked nature of the multi-block copolymer. Upon heating, a thermoplastic polymer becomes fluid, whereas it solidifies upon (re-)cooling. Thermoplastic polymers are soluble in proper solvents.

The term “hydrolysable” as used herein is meant to refer to the ability of reacting with water upon which the chemical bond is cleaved. Hydrolysable groups include ester, carbonate, phosphazene, amide, and urethane groups. Under physiological conditions, only ester, carbonate and phosphazene groups react with water in a reasonable time scale.

The term “multifunctional chain extender” as used herein is meant to refer to the presence of at least two reactive groups on the chain extender that allow chemically linking reactive pre-polymers thereby forming a multi-block copolymer.

The term “random multi-block copolymer” as used herein is meant to refer to a multi-block copolymer where the distinct segments are distributed randomly over the polymer chain.

The term “water-soluble polymer” as used herein is meant to refer to a polymer that has a good solubility in an aqueous medium, preferably water, under physiological conditions. This polymer, when copolymerised with more hydrophobic moieties, renders the resulting copolymer swellable in water. The water-soluble polymer can be derived from a diol, a diamine, or a diacid. The diol or diacid is suitably used to initiate the ring-opening polymerisation of cyclic monomers.

The term “swellable” as used herein is meant to refer to the uptake of water by the polymer. The swelling ratio can be calculated by dividing the mass of the water-swollen copolymer by the mass of the dry copolymer.

The term “semi-crystalline” as used herein is meant to refer to a morphology of the multi-block copolymer that comprises two distinctive phases, an amorphous phase and a crystalline phase. Preferably, the multi-block copolymer is made up of an amorphous phase and a crystalline phase.

### A1. Antibodies

The term “antibody” as used herein refers to an immunoglobulin molecule that is typically composed of two identical pairs of polypeptide chains, each pair of chain consist of one “heavy” chain with one “light” chain. The human light chains are classified as kappa and lambda. The heavy chains comprise different classes namely: mu, delta, gamma, alpha or epsilon. These classes define the isotype of the antibody, such as IgM, IgD, IgG, IgA and IgE, respectively. These classes are important for the function of the antibody and help to regulate the immune response. Both the heavy chain and the light chain consist of a variable and a constant region. The constant region of the heavy chain is clearly bigger than the constant region of the light chain, explaining the nomenclature of the heavy and light chain. Each heavy chain variable region (VH) and light chain variable region (VL) comprises complementary determining regions (CDR) interspersed by framework regions (FR). The variable region consists in total of four FRs and three CDRs. These are arranged from the amino- to the carboxyl-terminus as follows: FR1, CDR1, FR2, CDR2, FR3, CDR3, and FR4. The variable regions of the light and heavy chain together form the antibody binding site and defines the specificity for the epitope.

The term “antibody” as used herein encompasses murine, humanised, deimmunised human and chimeric antibodies, and an antibody that is a multimeric form of antibodies, such as dimers, trimers, or higher-order multimers of monomeric antibodies. Antibody also encompasses monospecific, bispecific or multi-specific antibodies, and any other modified configuration of the immunoglobulin molecule that comprises an antigen recognition site of the required specificity. The term antibody also encompasses an antibody that is linked or attached to a non-antibody moiety. Further, the term “antibody” is not limited by any particular method of producing the antibody. For example, it includes monoclonal antibodies (mAb), recombinant antibodies and polyclonal antibodies.

As used herein, antigen-binding fragments include Fab, F(ab′), F(ab′)₂, single-chain antibodies (scFv), and bivalent single-chain antibodies. In some instances, the term “antibody” as used herein is meant to also include an antigen binding fragment thereof. Hence, whenever the term “antibody” is used herein, it may be replaced by “an antigen binding fragment of an antibody”. In particular, antigen-binding fragments include such fragments that comprise at least two paired domains.

Antibodies as disclosed herein may further comprise a moiety for increasing the in vivo half-life of the molecule, such as but not limited to poly(ethylene glycol) (PEG), human serum albumin, glycosylation groups, fatty acids and dextran. Such further moieties may be conjugated or otherwise combined with the antibodies using methods well known in the art. In some embodiments, the antibodies as disclosed herein can be coupled to an active compound, for example a toxin. Furthermore, the antibodies or antigen binding fragments as disclosed may be coupled to a label, e.g. a fluorescent protein, chemical label, organic dye, coloured particle or enzyme. The antibodies as disclosed herein can be coupled to a drug to form an antibody-drug conjugate (ADC).

Preferably, an antibody as disclosed herein is a humanised antibody. The term “humanised antibody” refers to an antibody that contains some or all of the CDRs from a non-human animal antibody while the framework and constant regions of the antibody contain amino acid residues derived from human antibody sequences. Humanised antibodies are typically produced by grafting CDRs from a mouse antibody into human framework sequences followed by back substitution of certain human framework residues for the corresponding mouse residues from the source antibody. The term “deimmunised antibody” also refers to an antibody of non-human origin in which, typically in one or more variable regions, one or more epitopes have been removed, that have a high propensity of constituting a human T-cell and/or B-cell epitope, for purposes of reducing immunogenicity. The amino acid sequence of the epitope can be removed in full or in part. However, typically the amino acid sequence is altered by substituting one or more of the amino acids constituting the epitope for one or more other amino acids, thereby changing the amino acid sequence into a sequence that does not constitute a human T-cell and/or B-cell epitope. The amino acids are substituted by amino acids that are present at the corresponding position(s) in a corresponding human variable heavy or variable light chain as the case may be.

In some embodiments, an antibody as disclosed herein is a human antibody. The term “human antibody” refers to an antibody consisting of amino acid sequences of human immunoglobulin sequences only. Human antibodies may be prepared in a variety of ways known in the art.

In some embodiments, the antibody is an isolated antibody. The term “isolated” as used herein refer to material which is substantially or essentially free from components which normally accompany it in nature.

The antibodies disclosed herein can be produced by any method known to a skilled person. In some embodiments, antibodies may be prepared by immunising animals and collecting polyclonal antibodies or using standard hybridoma technology (Köhler et al., Nature 1975, 256(5517), 495-497). Antibodies may also be prepared using recombinant technology, for example by transfecting a host cell with nucleic acid expressing, for example, the respective heavy and light chains. Suitable cell lines are known to a skilled person and include a Chinese hamster ovary cell, an NS0 cell or a PER-C6 cell. The transfected cells are cultured and antibody is harvested from the culture medium. The antibody may be purified form the medium, preferably said antibody is affinity purified. Alternatively, said antibodies can be generated synthetically. Methods for preparing bispecific antibodies are also known in the art (see, e.g., WO-A-2013/157954).

The antibody preferably has a molecular weight of about 70 kDa or more, such as about 75 kDa or more, about 80 kDa or more, about 85 kDa or more, about 90 kDa or more, or about 100 kDa or more. Typically, the antibody or antigen binding fragment thereof can have a molecular weight of about 200 kDa or less, such as about 190 kDa or less, about 180 kDa, or less, about 170 kDa or less, about 160 kDa or less or about 150 kDa or less.

### A2. Large Proteins

As used herein, large proteins refer to proteins having a molecular weight of about 70 kDa or more, such as about 75 kDa or more, about 80 kDa or more, about 85 kDa or more, about 90 kDa or more, or about 100 kDa or more. Typically, the large proteins can have a molecular weight of about 200 kDa or less, such as about 190 kDa or less, about 180 kDa, or less, about 170 kDa or less, about 160 kDa or less or about 150 kDa or less. Preferably, the large protein is therapeutically active.

Some examples of large proteins that may be employed in the dosage forms of the invention include Fc-fusion proteins, antibody drug conjugates (ADCs), full length immunoglobulins, coagulation factors, growth factors, hormones, cytokines, enzymes and the like.

### B. Copolymer Microspheres

As used herein, the term “microsphere” refers to a spherical or spheroid particle about 999 μm or less in diameter in which may be loaded one or more therapeutic agents for drug delivery. In the context of this disclosure, a microsphere typically has a diameter of 1 μm or more. A microsphere can typically have a diameter of about 100 μm or less. Nonetheless, in some cases the microspheres can have a diameter of about 100 μm or more, such as about 200 μm or more, about 300 μm or more, or even up tot about 500 μm (for example, microparticles for tissue engineering applications to build a scaffold).

Aspects of the disclosure relate to microspheres formed by copolymers. In some embodiments, these copolymers may be selected based on the copolymer or a portion thereof having one or more of the following characteristics: (i) the polymer forms a matrix in which the antibody or protein can be incorporated, (ii) the polymer protects the antibody or protein from the environment in which it is stored and/or into which the microsphere is administered (e.g. temperature stability), (iii) the polymer is hydrophilic, (iv) the polymer enables diffusion of the antibody or protein, (v) the polymer will accommodate the hydrodynamic radius of the antibody or protein, (vi) the polymer is biodegradable, and/or (vii) the polymer degrades without impacting the purity of the antibody or protein.

The microsphere may release less than about 3% to about 40% of the antibody or protein contained therein based on total weight of the microsphere, within about 24 hours.

It is surprising that stable extended release formulations utilising polymers as described herein can be made as large therapeutic proteins, and in particular antibodies, are known to degrade (i) as a result of the harsh conditions (e.g. shear forces, oil-water interfaces, pH changes, etc.) used during the production of extended release formulations for antibodies or large therapeutic proteins, (ii) at elevated temperatures, such as elevated temperatures used during hot melt extrusion of extended release implants or body temperature, or (iii) in response to pH changes, such as pH changes caused by the acidic degradation products that are formed as a result of hydrolytic degradation of the polymers. Thus, prior to the present invention it was thought that it was not possible to achieve long term extended in vivo release of an antibody or large protein via the use of biodegradable extended release formulations comprising polymers as described herein. The present invention details the surprising discovery that biodegradable polymers as described herein can be used to form a stable extended release formulation of antibodies or large therapeutic proteins, where the structural integrity and biological activity of the antibody or protein is maintained during manufacturing and storage of the extended release formulation and where the antibody or protein is released in vivo structurally intact, properly folded and biologically active from the extended release formulation following administration into the body.

Microspheres comprising antibodies or large therapeutic proteins may be prepared by techniques known to those skilled in the art, including but not limited to, solvent evaporation and spray drying techniques. In some embodiments the microspheres are formed with a water-polymer ratio of between about 0.1 to about 1.0, such as but not limited to about 0.5 to about 1.0, about 0.55 to about 1.0, about 0.6 to about 1.0, about 0.65 to about 1.0, about 0.7 to about 1.0, about 0.75 to about 1.0, about 0.8 to about 1.0, about 0.85 to about 1.0, about 0.9 to about 1.0, or about 0.95 to about 1.0. In some embodiments, the water-polymer ratio of the microspheres is about 0.5, about 0.55, about 0.6, about 0.65, about 0.7, about 0.75, about 0.8, about 0.85, about 0.9, about 0.95, or about 1.0. These water polymer ratios may be adjusted to achieve a particular release profile based on the concentration of the antibody or protein and/or the polymer used to form the microsphere (Bos et al., Pharmaceutical Technology, October 2001, 110-120).

In some embodiments, the microspheres have a diameter of at least about 1 μm, at least about 2 μm, at least about 5 μm, at least about 10 μm, at least about 20 μm, at least about 30 μm, at least about 40 μm, at least about 50 μm, at most about 50 μm, at most about 60 μm, at most about 70 μm, at most about 80 μm, at most about 90 μm, at most about 100 μm, at most about 150 μm, at most about 200 μm, at most about 300 μm, or at most about 500 μm. In some embodiments, the microspheres have a diameter between about 15 μm and about 200 μm, between about 20 μm and about 150 μm, between about 25 μm and about 100 μm, between about 30 μm and about 80 μm, or between about 40 μm and about 100 μm.

In some embodiments, the concentration of the aqueous solution of antibody or protein used for the preparation of loaded microspheres is about 100 mg/ml or more, about 125 mg/ml or more, about 150 mg/ml or more, about 175 mg/ml or more, about 200 mg/ml or more, about 225 mg/ml or more, or about 250 mg/ml or more. The concentration of the aqueous solution of antibody or protein used for the preparation of loaded microspheres may be about 500 mg/ml or less, about 450 mg/ml or less, about 400 mg/ml or less, about 350 mg/ml or less, or about 300 mg/ml or less.

The microsphere may be a homogeneous or monolithic microsphere in which the antibody or protein is dispersed throughout the polymer matrix. It is also possible that the microsphere is of a reservoir type in which the antibody or protein is surrounded by a polymer in the mononuclear or poly-nuclear state.

The microspheres can be prepared by techniques known to those skilled in the art, including but not limited to coacervation, solvent extraction/evaporation, spray-drying and spray freeze-drying techniques.

For example, the microsphere can be prepared by a solvent extraction/evaporation technique which comprises dissolving the multi-block copolymer in an organic solvent, such as dichloromethane, and emulsification of the multi-block copolymer solution in an aqueous phase containing an emulsifying agent, such as polyvinyl alcohol (as described amongst other by Okada, Adv. Drug Del. Rev. 1997, 38(1), 43-70).

The characteristics, such as particle size, porosity and loading of the so-formed microspheres depend on the process parameters, such as viscosity or concentration of the aqueous polyvinyl alcohol phase, concentration of the multi-block copolymer solution, ratio of primary emulsion to polyvinyl alcohol phase and the stirring rate.

When the microspheres are formed by a spray-drying process, a low concentration of multi-block copolymer from about 0.5% to about 5% by total weight of the solution, in one embodiment about 2%, in the organic solvent, such as dichloromethane, is employed. Spray-drying results in general in the formation of porous, irregularly shaped particles.

As the microspheres are being formed, an antibody or large therapeutic protein is encapsulated in the microspheres or microparticles. In general, when the solvent extraction/evaporation technique is employed, the antibody or protein is first dissolved in the solution of the multi-block copolymer in an organic solvent, such as dichloromethane or ethyl acetate. The organic solution is then subsequently emulsified in an aqueous polyvinyl alcohol solution, which yields an oil-in-water (O/W) emulsion. The organic solvent is then extracted into the aqueous phase and evaporated to solidify the microspheres.

In general, when the solvent evaporation technique is employed, an aqueous solution of the antibody or protein is first emulsified in a solution of the multi-block copolymer in an organic solvent, such as dichloromethane. This primary emulsion is then subsequently emulsified in an aqueous polyvinyl alcohol solution, which yields a water-in-oil-in-water (W/O/W) emulsion. The organic solvent, such as dichloromethane or ethyl acetate, is then extracted similarly to the O/W process route to solidify the microspheres. Alternatively, water-soluble agents may be dispersed directly in a solution of the multi-block copolymer in an organic solvent. The obtained dispersion is then subsequently emulsified in an aqueous solution comprising a surfactant, such as polyvinyl alcohol, which yields a solid-in-oil-in-water (S/O/W) emulsion. The organic solvent is then extracted similarly to the O/W process route to solidify the microspheres.

Water-in-oil-in-oil (W/O/O) or solid-in-oil-in-oil (S/O/O) emulsification routes provide an interesting alternative to obtain microspheres with sufficient encapsulation efficiency. In the W/O/O process the antibody or protein is, similar to a W/O/W process, dissolved in an aqueous solution and emulsified with a solution of the polymer in an organic solvent, such as dichloromethane or ethyl acetate. Subsequently, a polymer precipitant. such as silicon oil, is then slowly added under stirring to form embryonic microparticles, which are then poured into heptane or hexane to extract the silicone oil and organic solvent and solidify the microparticles. The microparticles may be collected by vacuum filtration, rinsed with additional solvent and dried under vacuum. In the S/O/O emulsification route the antibody or protein is, similar to a S/O/W process, dispersed as a solid powder in a solution of the polymer in an organic solvent, such as dichloromethane or ethyl acetate. Subsequently, a polymer precipitant, such as silicon oil, is then slowly added under stirring to form embryonic microparticles, which are then poured into heptane or hexane to extract the silicone oil and dichloromethane and solidify the microparticles.

Stabilising agents may be added to the aqueous solution of the antibody or antigen binding fragment thereof to prevent loss of activity during processing into microspheres. Examples of such stabilising agents are polyvinyl alcohol, Tween®/polysorbate, human serum albumin, gelatine and carbohydrates, such as trehalose, inulin and sucrose.

When the spray-drying technique is employed, an aqueous solution of the antibody or antigen binding fragment thereof is emulsified in a solution of the copolymer in an organic solvent, such as dichloromethane, as hereinabove described. The water-in-oil emulsion is then spray-dried using a spray dryer.

### C. Multi-Block Copolymers

The copolymer is a block copolymer and may, optionally, comprise or alternatively consist essentially of, poly(ethylene glycol) (PEG) and one or more other polymers.

Exemplary polymers that can be utilised in the dosage forms for extended release of the invention include SynBiosys® polymers produced by Innocore Pharmaceuticals. InnoCore's SynBiosys® technology offers a platform of bioresorbable polymers that are specifically designed to function as drug delivery systems. These polymers are composed of D,L-lactide, L-lactide, glycolide, ε-caprolactone, p-dioxanone and/or poly(ethylene glycol), which monomers are used in numerous products, including biomedical implants, pharmaceutical drug delivery products and combination products that have been approved for use in humans. SynBiosys® polymers are described in WO-A-2013/015685, the complete content of which is herewith incorporated by reference. Further exemplary polymers that can be utilised in the dosage forms for extended release of the invention include the multi-block copolymers as described in non-prepublished European patent application No. 19200879.5, which is herewith also completely incorporated by reference.

The morphology and properties under physiological conditions (i.e. in the body) may be different from the morphology and properties under ambient conditions (dry, room temperature). The transition temperatures, T_{g} and Tₘ, as used herein, refer to the corresponding values of a material when applied in vivo; viz. when at equilibrium with an atmosphere that is saturated with water vapour and at body temperature. This may be simulated in vitro by performing DSC measurement after allowing the material to equilibrate with a water-saturated atmosphere.

Polymer matrix characteristics such as rate of controlled release, degradation, swelling and strength can be precisely controlled by the appropriate combination of the two copolymer segments.

The multi-block copolymers described herein are generally linear. However, it is also possible to prepare the copolymers in branched form. These non-linear copolymers may be obtained by using a multifunctional chain extender with more than two functional groups, for example a trifunctional chain extender such as a triisocyanate. Branched copolymers may show improved creep characteristics.

The multi-block copolymers used in the present invention have at least a (soft) pre-polymer (A) block and a (hard) pre-polymer (B) block. The pre-polymer (B) block may be based on poly(lactide) or on poly(p-dioxanone). These will now be discussed separately below.

The morphology of the multi-block copolymers is dependent on the environmental conditions: a DSC (Differential Scanning Calorimetry) measurement may be performed under inert (dry) conditions and the results may be used to determine the dry materials' thermal properties. However, the morphology and properties under physiological conditions (i.e. in the body) may be different from the morphology and properties under ambient conditions (dry, room temperature). It is to be understood that the transition temperatures, T_{g} and Tₘ as used herein refer to the corresponding values of a material when applied in vivo; viz. when at equilibrium with an atmosphere that is saturated with water vapour and at body temperature. This may be simulated in vitro by performing the DSC measurement after allowing the material to equilibrate with a water-saturated atmosphere.

The physicochemical properties (such as degradation, swelling and thermal properties) of the multi-block copolymers can be readily tuned by changing the type of monomers of the soft pre-polymer (A) segments and hard pre-polymer (B) segments and their chain length and chain ratio and by choosing the type and amount of chain extender.

The biodegradable multi-block copolymer comprises a biodegradable, phase separated, thermoplastic multi-block copolymer comprising at least one amorphous hydrolysable pre-polymer (A) segment and at least one semi-crystalline hydrolysable pre-polymer (B) segment, wherein
said multi-block copolymer under physiological conditions has a T_{g} of about 37° C. or less and a Tₘ of about 50° C. to about 110° C.;
the segments are linked by a multifunctional chain extender;
the segments are randomly distributed over the polymer chain; and
the pre-polymer (B) segment comprises a X—Y—X triblock copolymer wherein Y is a polymerisation initiator, and X is a poly(p-dioxanone) segment with a block length expressed in p-dioxanone monomer units of about 7 or more.

The inventors found that these multi-block copolymers with crystalline poly(p-dioxanone) building blocks yield dosage forms that surprisingly exhibit a faster degradation profile in vitro as compared to the above described multi-block copolymers with crystalline poly(lactide) building blocks. Multi-block copolymers with crystalline poly(lactide) building block have a degradation time of about 3-4 years. For the majority of sustained release drug delivery formulations, such a degradation time is undesirably long as it may lead to polymer accumulation upon repeated injection and could potentially induce long-term tolerability issues. The multi-block copolymers with crystalline poly(p-dioxanone) building blocks, on the other hand, exhibit a reduced in vitro degradation time of approximately 0.5-1.5 years, depending on the duration of release. At the same time, the degradation products of the multi-block copolymers do not, or hardly, lead to degradation of the antibody or protein. Hence, these biologically active compounds and their functionalities remain (or mainly remain) intact.

The multi-block copolymers have a T_{g} of about 37° C. or less under physiological conditions. This may be achieved by using a pre-polymer (A) with a T_{g} of about 37° C. or less under physiological conditions. Pre-polymer (A) can have a T_{g} of about 30° C. or less under physiological conditions, such as a T_{g} of about 25° C. or less, about 15° C. or less, or about 5° C. or less.

The pre-polymer (A) segment is derived from pre-polymer (A). Pre-polymer (A) is typically completely amorphous at physiological (body) conditions.

Pre-polymer (A) may e.g. be prepared by ring-opening polymerisation. Thus, a pre-polymer (A) may be a hydrolysable copolymer prepared by ring-opening polymerisation initiated by a diol or diacid compound. The diol compound can be an aliphatic diol or a low molecular weight polyether such as poly(ethylene glycol). The polyether is part of the pre-polymer (A) by using it as an initiator and it can additionally be mixed with pre-polymer (A), thus forming an additional hydrophilic segment. Pre-polymer (A) can comprise reaction products of ester forming monomers selected from diols, dicarboxylic acids, and hydroxycarboxylic acids. Pre-polymer (A) can comprise reaction products of cyclic monomers and/or non-cyclic monomers. Exemplary cyclic monomers include glycolide, L-lactide, D-lactide, D,L-lactide, ε-caprolactone, δ-valerolactone, trimethylene carbonate, tetramethylene carbonate, 1,5-dioxepane-2-one, 1,4-dioxane-2-one (p-dioxanone) and/or cyclic anhydrides such as oxepane-2,7-dione. In one embodiment, ε-caprolactone is used.

In case pre-polymer (A) comprises poly(D,L-lactide), the L/D ratio of the lactide may be away from unity (other than 50/50). For instance, an L/D ratio between 85/15 and 15/85 gives a completely amorphous homopolymer. Furthermore, it is known that an excess of one isomer (L or D) over the other increases the T_{g} of the poly(D,L-lactide).

Furthermore, pre-polymer (A) can be based on (mixtures of) condensation (non-cylic) type of monomers such as hydroxyacids (e.g. lactic acid, glycolic acid, hydroxybutyric acid), diacids (e.g. glutaric, adipic, succinic or sebacic acid) and diols such as ethylene glycol, diethylene glycol, 1,4-butanediol or 1,6-hexanediol, forming ester and/or anhydride hydrolysable moieties.

At least part of the pre-polymer (A) segment can be derived from a water-soluble polymer, such as about 30% or more by total weight of pre-polymer (A), about 40% to about 95%, about 50% to about 90%, or about 60% to about 85%.

This water-soluble polymer may, for example, comprise one or more selected from the group consisting of polyethers, such as poly(ethylene glycol) (PEG), poly(tetramethylene oxide) (PTMO), poly(propylene glycol) (PPG), poly(vinylalcohol) (PVA), poly(vinylpyrrolidone) (PVP), poly(vinylcaprolactam), poly(hydroxyethylmethacrylate) (poly-(HEMA)), poly(phosphazenes). poly(orthoesters), poly(orthoesteramides), or copolymers of any of these polymers. Preferably, the water-soluble polymer comprises one or more selected from the group of poly(ethylene glycol), poly(tetramethylene oxide), poly(propylene glycol), poly(vinylalcohol), poly(vinylpyrrolidone), and poly(vinylcaprolactam). More preferably, the water-soluble polymer comprises, or is, poly(ethylene glycol).

Some non-limiting examples of suitable pre-polymer (A) segments include poly(ε-caprolactone)-co-PEG-co-poly(ε-caprolactone), poly(D,L-lactide)-co-PEG-co-poly(D,L-lactide), poly(glycolide)-co-PEG-co-poly(glycolide), and poly(p-dioxanone)-co-PEG-co-poly(p-dioxanone).

In addition, the pre-polymer (A) segment may, at each side of the water-soluble polymer, comprise any copolymer of the above-mentioned monomers. Some non-limiting examples of such pre-polymer (A) segments include [poly(ε-caprolactone-co-D,L-lactide)]-co-PEG-co-[poly(ε-caprolactone-co-D,L-lactide)], [poly(s-caprolactone-co-glycolide)]-co-PEG-co-[poly(ε-caprolactone-co-glycolide)], [poly(ε-caprolactone-co-p-dioxanone)]-co-PEG-co-[poly(ε-caprolactone-co-p-dioxanone)], [poly D,L-lactide-co-glycolide)]-co-PEG-co-[poly(D,L-lactide-co-glycolide)], [poly D,L-lactide-co-p-dioxanone)]-co-PEG-co-[poly(D,L-lactide-co-p-dioxanone)], and [poly(glycolide-co-p-dioxanone)]-co-PEG-co-[poly(glycolide-co-p-dioxanone)],

Pre-polymer (A) can further comprise p-dioxanone. Such introduction of p-dioxanone monomers in the pre-polymer (A) segment can introduce additional crystallinity in the multi-block copolymers. The content of such p-dioxanone monomers in pre-polymer (A) can be about 80% or less by total weight of pre-polymer (A), such as about 60% or less, about 50% or less, about 40% or less, about 30% or less, about 20% or less, about 10% or less, or about 5% or less. The content of p-dioxanone monomers in pre-polymer (A) can be about 0.1% or more by total weight of pre-polymer (A), such as about 1% or more, or about 2% or more.

The pre-polymer (A) segment can have a number average molecular weight Mₙ of about 500 g/mol or more, such as about 1000 g/mol or more, about 1500 g/mol or more, or about 2000 g/mol or more. Typically, the pre-polymer (A) segment has a number average molecular weight Mₙ of about 10 000 g/mol or less, such as about 9000 g/mol or less, or about 8000 g/mol or less.

The content of the pre-polymer (A) segment in the copolymer can be about 5% to about 95% by total weight of the multi-block copolymer, such as about 10% to about 90%, about 30% to about 75%, or about 50% to about 70%.

The multi-block copolymers have a Tₘ of about 50° C. to about 110° C. under physiological conditions, such as in the range of about 60° C. to about 110° C., about 60° C. to about 100° C., about 70° C. to about 100° C., or about 70° C. to about 90° C. This is due to the pre-polymer (B) segment. Pre-polymer (B) may have a Tₘ in the range of about 60° C. to about 100° C., preferably in the range of about 75° C. to about 95° C. Pre-polymer (B) may have a T_{g} of about 0° C. or less, such as about −5° C. or less, −10° C. or less, −15° C. or less, or −20° C. or less.

The pre-polymer (B) segment comprises about 70% or more of poly(p-dioxanone) by total weight of said pre-polymer (B) segment. The pre-polymer (B) segment can comprise about 80% or more of poly(p-dioxanone) by total weight of the pre-polymer (B) segment, such as about 85% or more, about 90% or more, or about 95% or more. The pre-polymer (B) segment is based on a pre-polymer consisting of poly(p-dioxanone). The amorphous phase of the phase separated multi-block copolymers predominantly consists of the soft pre-polymer (A) segments.

Apart from poly(p-dioxanone), the pre-polymer (B) segment can comprise further monomer units such as ε-caprolactone and/or 6-valerolactone.

The pre-polymer (B) segment comprises an X—Y—X triblock copolymer, wherein Y is a polymerisation initiator and X is a poly(p-dioxanone) segment. The block length of the poly(p-dioxanone) segment X expressed in terms of p-dioxanone monomer units is about 7 or more. Suitably, the block length of the poly(p-dioxanone) segment X can be about 7 to about 35, such as about 8 to about 30, about 9 to about 25, about 10 to about 20, or about 12 to about 15.

If the block length of the pre-polymer (B) segment is too small, then the melting enthalpy is relatively low and crystallisation of the polymer matrix during extraction of dichloromethane will be slow and incomplete. This, in turn, results in slow and insufficient hardening of the microspheres due to which they can become sticky and lead to agglomeration and smearing of the microspheres during production resulting in a microsphere dry powder with a very broad particle size distribution and/or poor powder flowability. Furthermore, slow and incomplete crystallisation caused by a small block length of pre-polymer (B) segment may lead to extensive loss of protein and/or antibody or antigen binding fragment thereof from the microspheres during the extraction process. leading to poor encapsulation and low contents of protein and/or antibody or antigen binding fragment thereof in the resulting microspheres. Additionally, slow and incomplete crystallisation caused by a small block length of pre-polymer (B) segment gives rise to an instable product, as further crystallisation can take place during storage, thereby changing the critical properties of the product (such as the release rate).

The minimum length of the crystallisable pre-polymer (B) segment thus plays an important role in obtaining multi-block copolymers that combine good product stability with good processability. Proper microspheres cannot be made using multi-block copolymers where the pre-polymer (B) segment comprises a X—Y—X triblock copolymer where X is composed of a short poly(p-dioxanone) block, since the short poly(p-dioxanone) blocks do not sufficiently crystallise and/or crystallise very slowly. Such an incompletely crystallised polymer is instable upon storage as further crystallisation may occur. This in turn changes the critical properties of the polymer. Additionally, short pre-polymer (B) segments give rise to sticky polymers which are difficulties during processing such as agglomerate and fusing together of microspheres during the extraction/evaporation process step.

The polymerisation initiator Y in the X—Y—X triblock copolymer can suitably be a diol, such as an aliphatic diol with about 2 to about 8 carbon atoms. Examples of suitable aliphatic diols to be used as polymerisation initiator Y include ethylene glycol, 1,2-propanediol, 1,3-propanediol, 1,4-butanediol, 1,3-butanediol, 2,3-butanediol, diethylene glycol, dipropylene glycol, triethylene glycol, poly(ethylene glycol), 1,5-pentanediol, 1,6-hexanediol, neopentyl glycol, hydrogenated bisphenol A, and glycerol. Preferred polymerisation initiators include ethylene glycol, 1,2-propanediol, 1,3-propanediol, 1,4-butanediol, 1,3-butanediol, 2,3-butanediol, 1,5-pentanediol, and 1,6-hexanediol. More preferred polymerisation initiators include ethylene glycol, 1,4-butanediol and 1,6-hexanediol.

The pre-polymer (B) segment may have number average molecular weight Mₙ of about 1300 g/mol or more, such as about 1500 g/mol or more, about 2000 g/mol or more, about 2200 g/mol or more, or about 2500 g/mol or more. The number average molecular weight Mₙ of the pre-polymer (B) segment may be about 7200 g/mol or less, such as about 5000 g/mol or less, about 4500 g/mol or less, about 4000 g/mol or less, or about 3200 g/mol or less.

The pre-polymer (B) segment may have weight average molecular weight M_{w} of about 1800 g/mol or more, such as about 2100 g/mol or more, about 2600 g/mol or more, or about 3000 g/mol or more. The weight average molecular weight M_{w} of the pre-polymer (B) segment may be about 10 080 g/mol or less, such as about 7000 g/mol or less, about 6300 g/mol or less, about 5600 g/mol or less, or about 4200 g/mol or less.

The pre-polymer (B) segment may have a molecular weight distribution M_{w}/Mₙ of about 1.0 or more, such as about 1.1 or more, about 1.2 or more, about 1.3 or more, or about 1.4 or more. The molecular weight distribution of the pre-polymer (B) segment is typically about 3.0 or less, such as about 2.0 or less, about 1.8 or less, about 1.6 or less, about 1.5 or less, or about 1.4 or less.

Pre-polymer (B) can have a density (as measured according to ASTM D1505) of about 1.1 g/cm³ or more, such as about 1.15 g/cm³ or more, or about 1.2 g/cm³ or more. The density of pre-polymer (B) can be about 1.5 g/cm³ or less, such as about 1.45 g/cm³ or less, or about 1.4 g/cm³ or less.

The content of the pre-polymer (B) segment in the copolymer may be about 5% to about 95% by total weight of the multi-block copolymer, such as about 10% to about 90%, about 25% to about 70%, or about 30% to about 50%. Such contents generally result in desired materials with good physical (e.g. swelling) and degradation properties at the temperature of application (viz. about 37° C. for medical applications).

The multifunctional chain extender can be a difunctional aliphatic chain extender, preferably a diisocyanate, such as 1,4-butane diisocyanate or 1,6-hexane diisocyanate. Choosing the type and amount of chain extender is a way to customise the polymer properties. For example, the chain extender may act as a softener or it may affect the degree of phase separation.

In an embodiment, the biodegradable multi-block copolymer is a [poly(ε-caprolactone)-co-poly(ethylene glycol)-co-poly(ε-caprolactone)]-b-[poly(p-dioxanone)] multi-block copolymer.

The multi-block copolymers with crystalline poly(p-dioxanone) building blocks may also be represented by [(R¹R²ₙR³)_{q}]ᵣ[(R⁴ₚR⁵R⁶ₚ)]ₛ, wherein R¹, and R³ are independently selected from the group consisting of

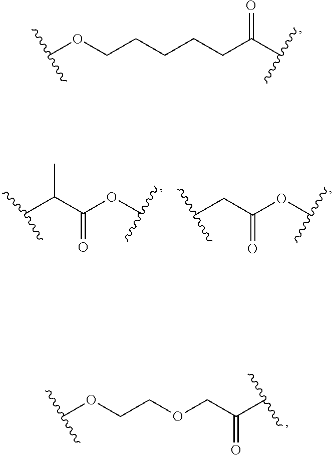

and any combination thereof,

### R² is

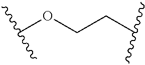 [SMILES: *CCO*]

and
R⁴ and R⁶ are each

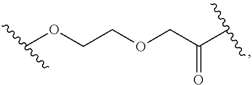 [SMILES: *OCCOCC(*)=O]

n, being the number of repeating R² moieties, is about 4 to about 120, preferably about 13 to about 70, more preferably about 20 to about 46;
p, being the number of repeating R⁴ and R^{e} moieties is about 7 or more, preferably about 7 to about 35, more preferably about 10 to about 20, even more preferably about 11 to about 14;
q, being the number average molecular weight of the (R¹R²ₙR³) block is about 400 g/mol to about 10 000 g/mol, preferably about 1000 g/mol to about 6000 g/mol, more preferably about 1400 g/mol to about 4000 g/mol, even more preferably about 1600 g/mol to about 3000 g/mol, most preferably about 1800 to about 2200 g/mol; and
r/s, being the ratio of pre-polymer (A) segment over pre-polymer (B) segment is about 0.1 to about 2.5.

In certain aspects.

n is about 20 to about 115, preferably about 35 to about 100, more preferably about 45 to about 85;
p is about 7 or more, preferably about 7 to about 35, more preferably about 10 to about 20, even more preferably about 10 to about 14;
q is about 1000 g/mol to about 7000 g/mol, preferably about 3000 g/mol to about 5000 g/mol, more preferably about 3800 g/mol to about 4200 g/mol; and
r/s is about 0.10 to about 1.0, such as about 0.15 to about 0.50, or about 0.20 to about 0.30.

When a PEG polymer is present in the multi-block copolymers with crystalline poly(p-dioxanone) building blocks, the length of the PEG may be varied from between about 1000 g/mol to about 5000 g/mol. Non-limiting examples include PEG lengths of at least about 1000 g/mol, at least about 1200 g/mol, at least about 1400 g/mol, at least about 1600 g/mol, at least about 1800 g/mol, at least about 2000 g/mol, at least about 2200 g/mol, at least about 2400 g/mol, at least about 2600 g/mol, at least about 2800 g/mol, at least about 3000 g/mol, at least about 3200 g/mol, at least about 3400 g/mol, at least about 3600 g/mol, at least about 3800 g/mol, at most about 4000 g/mol, at most about 4200 g/mol, at most about 4400 g/mol, at most about 4600 g/mol, at most about 4800 g/mol, or at most about 5000 g/mol.

### D. Formulations

Aspects of the disclosure relate to formulations comprising a plurality of microspheres. Such formulations may be comprised of a homogeneous or heterogeneous mixture of microspheres according to any one of the parameters disclosed herein. Further such formulations may optionally further comprise a pharmaceutically acceptable excipient and/or other components related to the specific indication being treated.

### E. Methods of Administration and Release Profile

A feature of the microspheres disclosed herein is a release profile that allows for extended release of antibody or protein. In some aspects, less than or about 1/7 of the antibody or protein in the microsphere or microsphere formulation is released in the first 24 hours post administration, such as less than or about 1/14, less than or about 1/21, less than or about 1/28, less than or about 1/29, less than or about 1/30, less than or about 1/31, less than or about 1/33, less than or about 1/34, less than or about 1/35, less than or about 1/42, less than or about 1/49, less than or about 1/56, less than or about 1/57, less than or about 1/58, less than or about 1/59, less than or about 1/60, less than or about 1/61, or less than or about 1/62. In some aspects, between about 2% to about 40% of the antibody or protein in the microsphere or microsphere formulation is released in the first 24 hours, for example about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%, or any range comprising two of these values, e.g., about 18.5% to about 26%.

Without wishing to be bound by any theory, it is envisioned that the release profiles described above allow for extended release of antibody or protein for specified periods, such as, but not limited to, about one week or more, about two weeks or more, about three weeks or more, about four weeks or more, about five weeks or more, about six weeks or more, about seven weeks or more, about eight weeks or more, about 12 weeks or more, about 16 weeks or more, about 20 weeks or more, about 24 weeks or more, about 28 weeks or more, about one month or more, about two months or more, about three months or more, about four months or more, about five months or more, about six months or more.

It is envisioned that the microspheres and formulations comprising these microspheres may be administered according to any mode of administration known in the art, including but not limited to topical, parenteral, oral, sublingual, via inhalation, nasal, via injection, intradermal, transdermal, intramuscular, subcutaneous. intravitreal, intra-articular, intra-arterial, intratumoral, bolus dose, infusion, and/or any other suitable method.

For topical administration, the microspheres may be contained in a gel, a cream or an ointment and may, if desired, be covered by a barrier. For example, the microspheres may be contained in a gel, such as a hyaluronic acid gel or a macromolecular polysaccharide gel.

When administered via injection, the microspheres may be contained in a pharmaceutical carrier, such as water, saline solution (for example 0.9%), or a solution containing a surfactant in an amount of from about 0.1% to about 0.5% w/v. An example of a surfactants which may be employed is Tween 80 surfactant. The pharmaceutical carrier may further contain a viscosifier, such as sodium carboxymethylcellulose.

In certain aspects, the disclosure relates to methods of administration that achieve or approximate sustained release profile with minimal or no burst release.

Aspects of the disclosure relate to regimens of administration that allow the maintenance of therapeutically relevant plasma levels. Such regimens include administration of the dosage form for extended release of antibody or protein about every one week, two weeks, three weeks, four weeks, five weeks, six weeks, seven weeks, eight weeks, 12 weeks, 16 weeks, weeks, 24 weeks, 28 weeks, or about every one month, two months, three months, four months, five months, six months.

### F. Methods of Treatment

The dosage forms disclosed herein may be used to treat a variety of diseases and may be administered according to appropriate periodicity and dose based on the indication. Indications may be human or veterinary. Exemplary indications include different cancers, acute myeloid leukaemia, non-Hodgkin's lymphoma, rheumatoid arthritis, multiple sclerosis, cardiovascular diseases, systemic lupus erythematosus. Crohn's disease, ulcerative colitis. Psoriasis, atopic dermatitis, transplant rejections, Alzheimer's disease, inflammatory diseases, viral infections, like hepatitis C infection, Ebola or HIV, ankylosing spondylitis, macular degeneration, allergic asthma, Migraine headache, Haemophilia A, Hypercholesterolemia, and several more conditions and/or any other disease or disorder associated with antibodies or therapeutic proteins indicated for oncology, hematology, cardiology/vascular disease, dermatology, endocrinology, gastroenterology, genetic disease, immunology, infectious diseases, musculoskeletal, nephrology, ophthalmology, pulmonary/respiratory disease, and rheumatology. These examples are only intended to give an enumeration of diseases for which a person may receive antibodies or therapeutic proteins. This list is not an exhaustive list. In addition, new indications for both existing and new antibodies and therapeutic proteins against additional target antigens are expected to be developed and found as disease mechanisms are elucidated and antigens for which new drugs are being developed are identified. Accordingly, these, not listed here, antibodies or therapeutic proteins can be formulated in the same manner as described here.

Further, unlike methods disclosed in the art for administration of an antibody or a protein, the antibody or protein containing microspheres and formulations disclosed herein require fewer administrations, e.g. less than about 10, less than about 9, less than about 8, less than about 7, less than about 6, less than about 5, less than about 4, less or than about 3 administrations, or about 1 or about 2 administrations, vs. 45 administrations of conventional antibodies over 3 or 6 months. In some embodiments, the described formulations are in the form of one injection providing a therapeutic effect longer than one month.

### G. General Definitions

As used in the description of the invention and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

As used herein, the term “comprising” is intended to mean that the formulations and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising”. “Consisting of” shall mean excluding more than trace elements of other ingredients and substantial method steps for administering the formulations disclosed herein. Aspects defined by each of these transition terms are within the scope of the present disclosure.

A “formulation” is intended to mean a combination of active agent and another compound or composition, inert (for example, a detectable agent or label) or active, such as an adjuvant.

A “pharmaceutical formulation” is intended to include the combination of an active agent with a carrier, inert or active, making the formulation suitable for diagnostic or therapeutic use in vitro, in vivo or ex vivo.

“Pharmaceutically acceptable carriers” refers to any diluents, excipients, or carriers that may be used in the formulations of the invention. Pharmaceutically acceptable carriers include ion exchangers, alumina, aluminium stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes. such as protamine sulphate, disodium hydrogen phosphate, potassium hydrogen phosphate, sodium chloride, zinc salts, colloidal silica, magnesium trisilicate, polyvinyl pyrrolidone, cellulose-based substances, poly(ethylene glycol), sodium carboxymethylcellulose, polyacrylates, waxes, polyoxyethylene-polyoxypropylene-block polymers, and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like, and consistent with conventional pharmaceutical practices.

The term “extended release” is used herein to refer to the ability to release an ingredient over a specified period of time. The term “burst release” refers to a phase of rapid release of the ingredient into the environment, such that continued release over an extended period of time is not sustainable.

As used herein, the terms “subject” or “patient” are used interchangeably to mean any animal. In some embodiments, the subject may be a mammal; in further embodiments, the subject may be a human, mouse, or rat.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. For instance, descriptors may be used to refer to biological material (e.g. tissue, organoids, samples) exhibiting characteristics of a particular organ, e.g. the use of “hepatic” to describe liver-derived tissue or a liver-like organoid. While not explicitly defined by below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques, which are within the skill of the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

### V. EXAMPLES

The following examples are non-limiting and illustrative of procedures which can be used in various instances in carrying the disclosure into effect. Additionally, all references disclosed herein are incorporated by reference in their entirety.

### Example 1—Synthesis of SynBiosys Multi-Block Copolymers

This example describes the synthesis and characterization of [poly(ε-caprolactone)-co-poly(ethylene glycol)-co-poly(ε-caprolactone)]-b-[poly(L-lactide)] and [poly(ε-caprolactone)-co-poly(ethylene glycol)-co-poly(ε-caprolactone)]-b-[poly(p-dioxanone)] multi-block copolymers used in the preparation of mAb-loaded microspheres.

Poly(ε-caprolactone)-co-PEG3000-co-poly(s-caprolactone) pre-polymer with a molecular weight (Mₙ) of around 4000 g/mol (abbreviated as PCL-PEG3000-PCL or CP30C40) and poly(ε-caprolactone)-co-PEG1000-co-poly(ε-caprolactone) pre-polymer with Mₙ of around 2000 g/mol (abbreviated as PCL-PEG1000-PCL or CP10C20) were prepared by ring-opening polymerisation of s-caprolactone using poly(ethylene glycol) with a molecular weight of 3000 g/mol (PEG3000) or 1000 g/mol (PEG1000) as initiator and stannous octanoate as a catalyst. Poly(L-lactide) pre-polymers with Mₙ of around 4000 g/mol (abbreviated as PLLA or LL40) were synthesised by ring-opening polymerisation of L-lactide using 1,4-butanediol as initiator and stannous octanoate as a catalyst. Molecular weights of the pre-polymers were analysed by ¹H-NMR. Poly(p-dioxanone) pre-polymers with Mₙ of around 2500 g/mol (abbreviated as PDO or D25) were synthesised by ring-opening polymerization of p-dioxanone using 1,4-butanediol as initiator and stannous octanoate as a catalyst.

[PCL-PEG3000-PCL]-b-[PDO], [PCL-PEG1000-PCL]-b-[PDO], [PCL-PEG3000-PCL]-b-[PLLA], and [PCL-PEG1000-PCL]-b-[PLLA] multi-block copolymers with a block ratio of 50/50 w/w were prepared by chain-extension of PCL-PEG3000-PCL or PCL-PEG1000-PCL pre-polymer with either PDO or PLLA pre-polymer in p-dioxane using 1,4-butanediisocyanate as a chain extender followed by precipitation to remove p-dioxane.

The polymers, abbreviated as 50CP30C40-LL40, 50CP10C20-LL40, 50CP30C40-D25 and 50CP10C20-D25 were analysed for their chemical composition, intrinsic viscosity, residual p-dioxane content, and thermal characteristics.

The chemical composition (monomer ratio) and molecular weight (Mₙ) of the prepolymers, as well as the block ratio of the multi-block copolymers was determined by ¹H-NMR. For this determination a Bruker Avance DRX 500 MHz NMR spectrometer (B AV-500) was used equipped with Bruker Automatic Sample Changer (BACS 60) (Varian) operating at 500 MHz. The di delay time was set to 20 s, and the number of scans was 16. Spectra were recorded from 0 to 14 ppm. ¹H-NMR samples were prepared by adding about 1.3 g of deuterated chloroform to about 25 mg of polymer.

Intrinsic viscosity was measured using an Ubbelohde Viscosimeter (DIN), type OC, Si Analytics supplied with a Si Analytics Viscosimeter including a water bath. The measurements were performed in chloroform at 25° C. The polymer concentration in chloroform was such that the relative viscosity was in the range of 1.2-2.0.

Residual p-Dioxane content was determined using a GC-FID headspace method. Measurements were performed on a GC-FID Combi Sampler supplied with an Agilent Column, DB-624/30 m/0.53 mm. Samples were prepared in DMSO (dimethylsulphoxide). Residual solvent content was determined using p-dioxane calibration standards.

Modulated differential scanning calorimetry (MDSC) was used to determine the thermal behaviour of the multi-block copolymers using a Q2000 MDSC (TA instruments, Ghent, Belgium). About 5-10 mg of dry material was accurately weighed and heated under a nitrogen atmosphere from −85° C. to 120° C. at a heating rate of 2° C./min and a modulation amplitude of +/−0.42° C. every 80 seconds. The glass transition temperature (T_{g}, midpoint), melting temperature (maximum of endothermic peak, Tₘ) and the melting enthalpy (ΔHₘ), which was calculated from the surface area of the melting endotherm, were determined from the reversing heat flow. Temperature and enthalpy were calibrated with an indium standard.

Table 1 lists the characteristics of the various multi-block copolymers.

TABLE 1
Characteristics of SynBiosys multi-block copolymers used for the
preparation of mAb-loaded microspheres.

| | | | PEG MW | IV | Residual dioxane | Tₘ | ΔHₘ |
|---|---|---|---|---|---|---|---|
| RCP | Polymer grade | Block ratio (-) | (g/mol) | (dl/g) | (ppm) | (° C.) | (J/g) |
| 1667 | 50CP30C40-LL40 | 48.3/51.7 | 2998 | 0.76 | <15 | 128 | 50 |
| 1677 | 50CP30C40-LL40 | 49.7/50.3 | 2998 | 0.78 | <15 | 127 | 40 |
| 1440 | 50CP10C20-LL40 | 47.0/53.0 | 1021 | 0.70 | 217 | 128 | 20 |
| 1915 | 50CP30C40-D27 | 49.7/50.3 | 3250 | 0.75 | <18 | 78 | 72 |
| 1914 | 50CP10C20-D27 | 49.9/50.1 | 1012 | 0.73 | <18 | 74 | 75 |

### Example 2—Production of SynBiosys mAbX Loaded Microspheres

A monoclonal antibody with a molecular weight of 145.8 kDa (mAbX) was formulated into SynBiosys microspheres. mAbX-loaded microspheres (mAbX MSP) with a target loading of 5 wt. % were prepared of blends of 50CP10C20-LL40 (RCP-1440) and 50CP30C40-LL40 (RCP1667) via water-in-oil-in oil (W/O/O) and water-in-oil-in-water (W/O/W)-based microencapsulation processes.

mAbX solution was concentrated to a target concentration of 100 mg/ml. Visual examination of the concentrated protein solution showed that there were no insoluble particles. The integrity of mAbX was maintained during concentration. SEC-UPLC analysis confirmed the absence of aggregates and there were no signs of degradation. The mAbX concentration of the concentrated solution was 105.7 mg/ml as determined with SEC-UPLC.

50CP10C20-LL40 and 50CP30C40-LL40 were dissolved in dichloromethane at different weight ratios (100:0, 50:50, 25:75 and 0:100) to a final polymer concentration of 10 wt. % and filtered through a 0.2 μm PTFE filter. 5.0 g of polymer solution (O₁) was then emulsified with 0.25 ml of concentrated mAbX solution (W1) at an O₁/W₁ ratio of 20 w/v using an Ultra Turrax (22 000 rpm for 40 seconds) to yield a primary emulsion (dispersed phase, DP).

For the preparation of mAbX MSP via the W/O/O microencapsulation route, 4.5 g of silicone oil, which serves as a coacervation agent, was subsequently added to DP under vortexing by means of a syringe pump to initiate dichloromethane extraction and form embryonic MSPs. Under constant stirring, the embryonic MSP were then transferred to a vessel containing 330 g heptane to extract dichloromethane and silicone oil from the embryonic MSPs. After completion of the extraction procedure, the microspheres were collected by filtration using a 5 μm filter, washed with heptane (2×250 ml) and finally dried overnight at 40° C. under reduced pressure.

For the preparation of mAbX MSP via the W/O/W microencapsulation route 5.0 ml DP was subsequently emulsified with 250 ml of a 0.4% aqueous PVA solution containing 5 wt. % NaCl (W₂ or CP) via membrane emulsification using a membrane with 30 μm pores thereby forming a water-in-oil-in-water (W/O/W) double emulsion. The W/O/W emulsion was stirred for 1.5 hours at room temperature to allow extraction and evaporation of dichloromethane. After completion of solvent evaporation, the mAbX MSP were collected by filtration, washed with 0.05 wt. % aqueous Tween-80 solution and water for injection and finally lyophilised.

The resulting microspheres were analysed for their particle size distribution using a Horiba LA-960 Laser Particle Size Analyser (Table 2).

TABLE 2
Particle size distributions of mAbX-loaded MSP prepared
via W/O/O and W/O/W microencapsulation route.

| | | Weight fraction | | | |
|---|---|---|---|---|---|
| | Microen- | 50CP30C40-LL40 and | | | |
| | capsu- | 50CP10C20-LL40 | | | |
| | lation | 50CP30C40- | 50CP10C20- | D50 | CV |
| Batch nr. | route | LL40 | LL40 | (μm) | (%) |
| AMD 16143 | W/O/O | 100% | 0% | 53 | 42 |
| AMD 16144 | W/O/O | 50% | 50% | 54 | 41 |
| AMD 16145 | W/O/W | 100% | 0% | 57 | 11 |
| AMD 16146 | W/O/W | 0% | 100% | 49 | 11 |
| AMD 16148 | W/O/O | 25% | 75% | 56 | 34 |
| AMD 16149 | W/O/O | 0% | 100% | 54 | 44 |
| AMD 16178 | W/O/W | 50% | 50% | 50 | 12 |
| AMD 16179 | W/O/W | 25% | 75% | 48 | 12 |

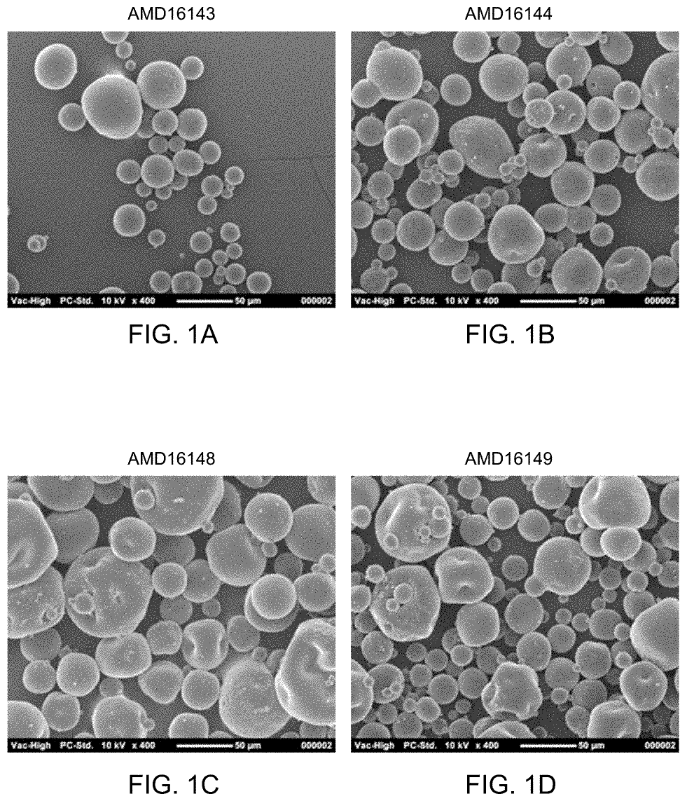

The average particle size of the mAbX MSP varied between 48 and 57 μm. mAbX MSP prepared via the W/O/O route had a relatively broad particle size distribution (FIG. 1) with coefficient of variance (CV) of 30 to 40% whereas mAbX MSP prepared via the W/O/W process route had a very narrow particle size distribution (CV 11-12%).

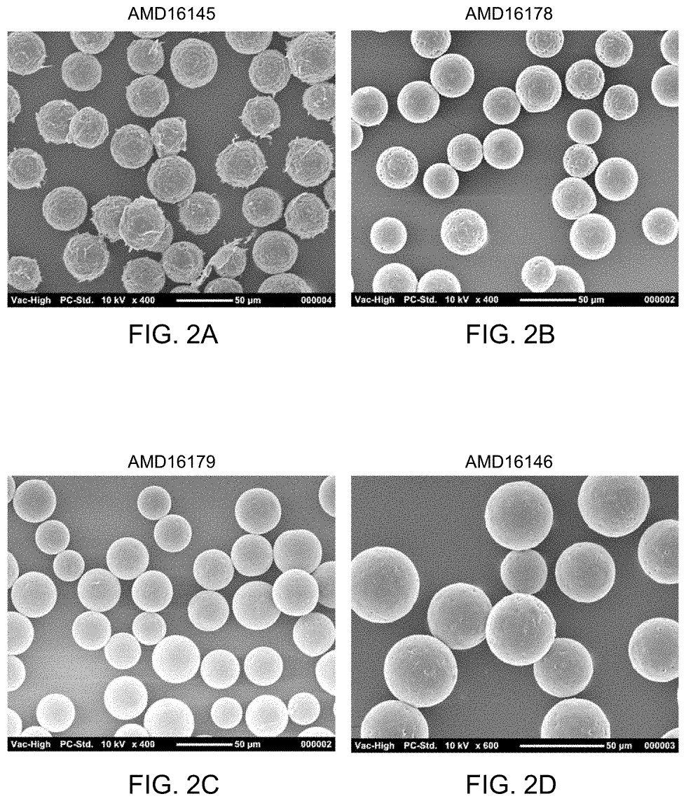

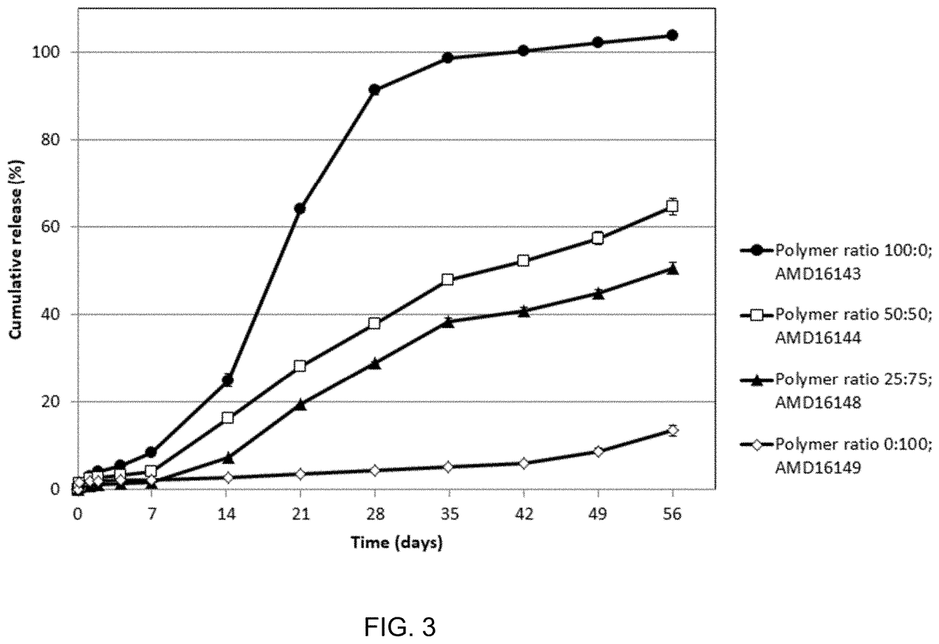

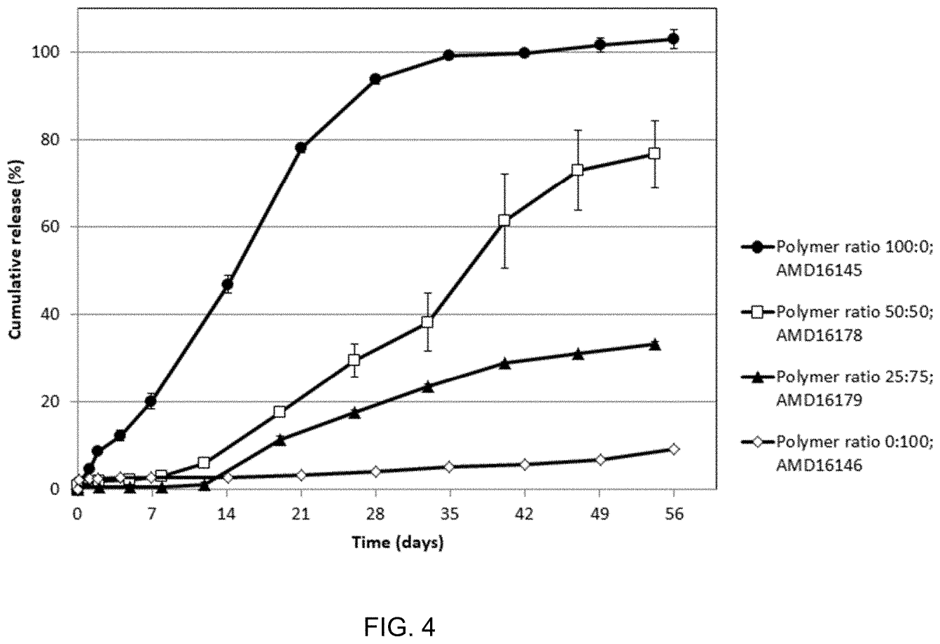

Scanning electron microscopy (SEM) using a JEOL JCM-5000 Neoscope SEM confirmed the results obtained with laser diffraction. mAbX MSP prepared with the W/O/O typically had a smooth surface with irregular raisin-like shapes and a broad particle size distribution (FIG. 1). The W/O/W microencapsulation process yielded uniformly sized spherical microspheres with a smooth surface (FIGS. 2 B-D), except for mAbX-MSP entirely composed of 50CP30C40-LL40 which exhibited a rough surface with fibre-like structures present at the surface (FIG. 2 A).

All mAbX MSP were analysed for their mAbX content. mAbX MSP (˜10 mg) were completely dissolved in 1.0 ml DMSO (80° C., 30 minutes) after which 5.0 ml of aqueous 0.1 N NaOH 0.5% SDS solution was added to completely hydrolyse the polymers (room temperature, overnight stirring). Total protein content of the clear solutions was then determined using a bicinchoninic acid (BCA) assay. The results presented in Table 3 show that both the coacervation-based W/O/O process as well as the W/O/W double emulsification process allow microencapsulation of mAbX at high encapsulation efficiencies (EE) of typically >90%, except for mAbX-MSP entirely composed of 50CP30C40-LL40, resulting in mAbX MSP with mAbX loading of 4.6-4.9 wt. %.

TABLE 3
mAbX content and encapsulation efficiency of mAbX-loaded MSP
prepared via W/O/O and W/O/W microencapsulation route.

| | | Weight fraction | | | |
|---|---|---|---|---|---|
| | Microen- | 50CP30C40-LL40 and | | mAbX | Encaps. |
| | capsu- | 50CP10C20-LL40 | | con- | effi- |
| | lation | 50CP30C40- | 50CP10C20- | tent | ciency |
| Batch nr. | route | LL40 | LL40 | (wt. %) | (%) |
| AMD 16143 | W/O/O | 100% | 0% | 4.8 | 95 |
| AMD 16144 | W/O/O | 50% | 50% | 4.8 | 95 |
| AMD 16145 | W/O/W | 100% | 0% | 3.8 | 77 |
| AMD 16146 | W/O/W | 0% | 100% | 4.9 | 98 |
| AMD 16148 | W/O/O | 25% | 75% | 4.9 | 98 |
| AMD 16149 | W/O/O | 0% | 100% | 4.8 | 95 |
| AMD 16178 | W/O/W | 50% | 50% | 4.9 | 98 |
| AMD 16179 | W/O/W | 25% | 75% | 4.6 | 92 |

mAbX in vitro release kinetics was determined by incubating 20 mg of mAbX MSP in 2 ml polypropylene vials containing 1.8 ml in vitro release buffer (100 mM phosphate buffer, pH 7.4 with 0.025% Tween 20 and 0.02% NaN₃) which were placed on an orbital shaker in a climate chamber thermostated at 37° C. At predetermined time points, following centrifugation of the vials, aliquots of 1.6 ml buffer were removed and replaced by fresh buffer. mAbX concentrations in the buffer were determined via SEC-UPLC with a Waters 2690 HPLC system using a TSKgel Size Exclusion HPLC column, 300×4.6 mm; 4 μm, isocratic analysis at 25° C. (50 mM phosphate, 0.4 M perchlorate buffer pH 6.3: acetonitrile (90:10, v/v), and UV-detection at 214 nm.

mAbX release kinetics were hardly affected by the type of microencapsulation process (W/O/O or W/O/W), but mainly by the polymer matrix composition. mAbX MSP prepared of 100% 50CP30C40-LL40 gradually and completely released mAbX over a one month period. By (partly) replacing 50CP30C40-LL40 by 50CP10C20-LL40 (and thereby lowering the swelling degree of the polymer matrix) the release rate of mAbX was effectively decreased. Based on extrapolation of the release curves. mAbX MSP prepared of a 50/50 blend of 50CP30C40-LL40 and 50CP10C20-LL40 release mAbX gradually with a total estimated duration of release of approximately 3 months, whereas mAbX MSP prepared of a 25/75 blend of 50CP30C40-LL40 and 50CP10C20-LL40 release mAbX gradually with a total estimated duration of release of 4 (W/O/W) to 5 (W/O/O) months.

### Example 3—Production of SvnBiosys mAbX MSP with High mAbX Loading Via W/O/W Microencapsulation

mAbX MSP with a target loading of 10-20 wt. % were manufactured at a scale of 1 g via the W/O/W-based membrane emulsification process described in Example 2. The resulting mAbX microspheres were characterized using the methods described in Example 2, except that their particle size distribution was measured using a Coulter Counter Multisizer III. The volume average particle size (D50) and coefficient of variation (CV) were determined in the range of 4-120 μm or 8-240 μm.

mAbX MSP with a target mAbX loading of 10 wt. % were initially prepared of 100% 50CP30C40-LL40 in an attempt to produce mAbX MSP with one month sustained release of mAbX. However. the combination of higher mAbX loading and relatively high swelling degree of 50CP30C40-LL40 resulted in a poor encapsulation efficiency of only 58% (AMD16172). By blending 50CP30C40-LL40 with 10 to 20 wt. % 50CP10C20-LL40 the encapsulation efficiency could be significantly improved (70-80%) yielding mAbX MSP with mAbX content of 7.0 wt. % (AMD17013) and 8.2 wt. % (AMD17005) (Table 4).

TABLE 4
Characteristics of mAbX-loaded MSP with 10% mAbX target
loading prepared via W/O/W microencapsulation route.

| | | Target | | | | D50 | mAbX | |
|---|---|---|---|---|---|---|---|---|
| | Polymer | mAbX | | O₁/W₁ | mAbX | (μm); | content | |
| | ratio | loading | Pol. Cone. | ratio | cone, | (CV, | (wt. | EE |
| Batch nr. | (w/w) | (wt. %) | (wt. %) | (w/v) | (mg/ml) | %) | %) | (%) |
| AMD16172 | 100:0 | 10 | 10 | 9.5 | 105.7 | 54(12) | 5.8 | 58 |
| AMD17005 | 80:20 | 10 | 10 | 9.5 | 105.7 | 49(11) | 8.2 | 82 |
| AMD17013 | 90:10 | 10 | 10 | 9.5 | 105.7 | 64(17) | 7.0 | 70 |

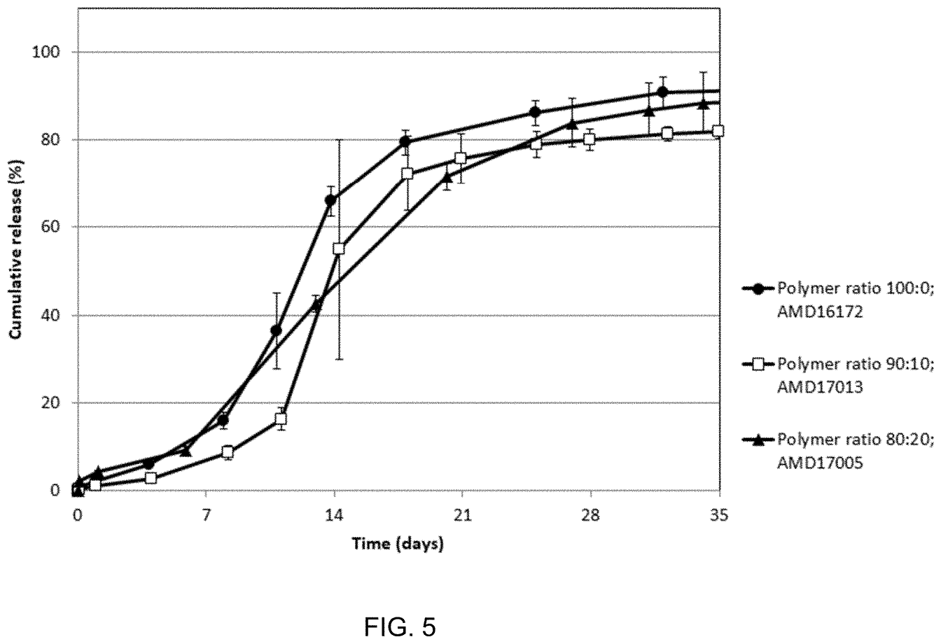

FIG. 5 shows that the cumulative release kinetics of mAbX MSP prepared of a 90:10 polymer ratio were similar to mAbX MSP completely composed of 50CP30C40-LL40 showing sigmoidal release kinetics and complete release in 2 to 3 weeks. By increasing the weight fraction 50CP10C20-LL40 to 20 wt. %, the release of mAbX could be slowed down.

To allow the preparation of mAbX MSP with a target loading of 19 wt. %, the mAbX solution was further concentrated to ˜230 mg/ml, after which mAbX MSP were prepared of blends of 50CP30C40-LL40 and 50CP10C20-LL40 using the W/O/W process as described above using the settings listed in Table 5. Unfortunately, the majority of the so-prepared mAbX MSP formulations had very poor encapsulation efficiencies, irrespective of polymer ratio, polymer concentration. and primary emulsification conditions. Significantly higher encapsulation efficiencies (70-75%) were obtained for mAbX MSP with a target mAbX loading of 14-15%, that were prepared using more concentrated polymer solutions (15 wt. %) yielding mAbX MSP with ˜10.6 wt. % mAbX (AMD17042 and AMD17038) (fable 5).

TABLE 5
Characteristics of mAbX- loaded MSP with 15-20% mAbX target
loading prepared via W/O/W microencapsulation route.

| | Polymer | Target mAbX | Pot | O₁/W₁ | mAbX | D50 | mAbX | |
|---|---|---|---|---|---|---|---|---|
| | ratio | loading | Conc. | ratio | conc. | (m); | content | EE |
| Batch nr. | (w/w) | (wt. %) | (wt. %) | (w/v) | (mg/ml) | (CV, %) | (wt. %) | (%) |
| AMD17016 | 80:20 | 19 | 10 | 9.5 | 222.8 | 60(18) | 1.0 | 5 |
| AMD17017 | 90:10 | 19 | 10 | 9.5 | 222.8 | 70(18) | 7.9 | 41 |
| AMD17021 | 90:10 | 19 | 10 | 9.50 | 222.8 | 70(18) | 7.9 | 41 |
| AMD17052 | 90:10 | 17.3 | 12.5 | 9.5 | 248.0 | 56(13) | 2.7 | 16 |
| AMD17051 | 90:10 | 14.9 | 15 | 9.5 | 248.0 | 58(13) | 2.0 | 14 |
| AMD17041 | 80:20 | 20.7 | 10 | 9.5 | 248.0 | 53(10) | 5.2 | 25 |
| AMD17042 | 80:20 | 14.9 | 15 | 9.5 | 248.0 | 64(10) | 10.6 | 71 |
| AMD17043 | 70:30 | 20.7 | 10 | 9.5 | 248.0 | 49(10) | 5.4 | 26 |
| AMD17034 | 90:10 | 19.4 | 10 | 9.5 | 228.3 | 54(15) | 4.6 | 24 |
| | | | | | (50 mM A)^{c} | | | |
| AMD17035 | 90:10 | 19.9 | 10 | 9.5 | 236.0 | 47(11) | 3.2 | 16 |
| | | | | | (150 mM A)^{c} | | | |
| AMD17036 | 90:10 | 20.1 | 10 | 9.3 | 236.0 | 57(12) | 8.9 | 44 |
| | | | | | (150 mM A)^{c} | | | |
| AMD17038 | 90:10 | 14.3 | 15 | 9.5 | 236.0 | 65(12) | 10.7 | 75 |
| | | | | | (150 mM A)^{c} | | | |

^{a}weight ratio of 50CP30C40-LL40 and 50CP10C20-LL40;
^{b}DP prepared via 2 × 40 sec emulsification, Ultraturrax, 21 600 rpm;
^{c}mAbX solution contained 50 or 150 mM L-arginine, 0.025% (w/v) Tween 80.

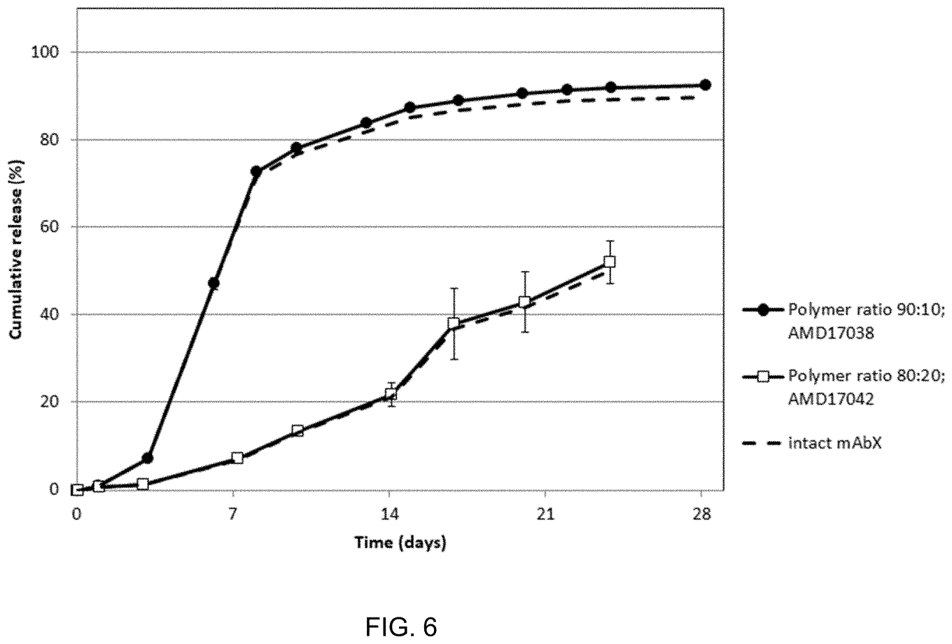

AMD17042 and AMD17038 were analysed for their in vitro release kinetics (FIG. 6). mAbX release from the mAbX MSP prepared of 90:10 w/w blends of 50CP30C40-LL40 and 50CP10C20-LL40 (AMD17038) was in line with release kinetics previously generated for mAbX MSP prepared of the same polymer matrix but with lower mAbX content (e.g. AMD17013). Surprisingly, mAbX release from the mAbX MSP prepared of 80:20 w/w blends of 50CP30C40-LL40 and 50CP10C20-LL40) was significantly slower showing near-linear release of mAbX for the first 4 weeks with an estimated duration of release of approximately 2 months (based on extrapolation of the data). Both AMD17042 and AMD17038 released highly intact mAbX (integrity >96% as determined via SEC-UPLC).

### Example 4—Production of SynBiosys mAbX Microspheres with High mAbX Loading Via W/O/O Microencapsulation

To allow the preparation of mAbX MSP with mAbX loading of ˜20 wt. % the W/O/W microencapsulation process was replaced by the water-in-oil-in-oil (W/O/O) based microencapsulation process used in Example 2. In the W/O/O-based microencapsulation process, extraction of dichloromethane is performed using a water free extraction process that prevents the loss of water soluble API during extraction of dichloromethane and hardening of the microspheres, thereby allowing high encapsulation efficiency of ˜100%.

mAbX MSP with a target loading of 10 and 19 wt. % were prepared of the 90/10 blend of 50CP10C40-LL40 and 50CP10C20-LL40 via the W/O/O microencapsulation process described in Example 2 using the settings listed in Table 6.

TABLE 6
Characteristics of mAbX-loaded MSP prepared via W/O/O
microencapsulation route.

| | | Target. | | | | | | |
|---|---|---|---|---|---|---|---|---|
| | Polymer | mAbX | | 0₁/W₁ | mAbX | | mAbX | |
| | ratio | loading | Pol. Conc. | ratio | conc. | D50 (μm); | content | EE |
| Batch nr. | (w/w) | (wt. %) | (wt. %) | (w/v) | (mg/ml) | (CV, %) | (wt. %) | (%) |
| AMD17022 | 90:10 | 10 | 10 | 9.5 | 105.7 | 78(66) | 9.3 | 93 |
| AMD17023 | 90:10 | 19 | 10 | 9.5 | 222.8 | 109(81) | 19.0 | 100 |

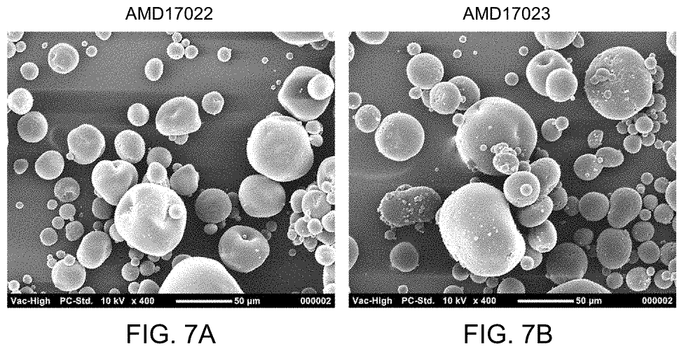

The average particle size of the mAbX MSP (as measured by laser diffraction) was relatively high with D50 of 78 and 109 μm, respectively. Both formulations had a very broad particle size distribution with CV values of 66 to 81%. SEM confirmed the results obtained with laser diffraction (FIG. 7). As expected the encapsulation efficiency was very high (>90%) yielding mAbX MSP with mAbX loading of 9.3 and 19.0 wt. %.

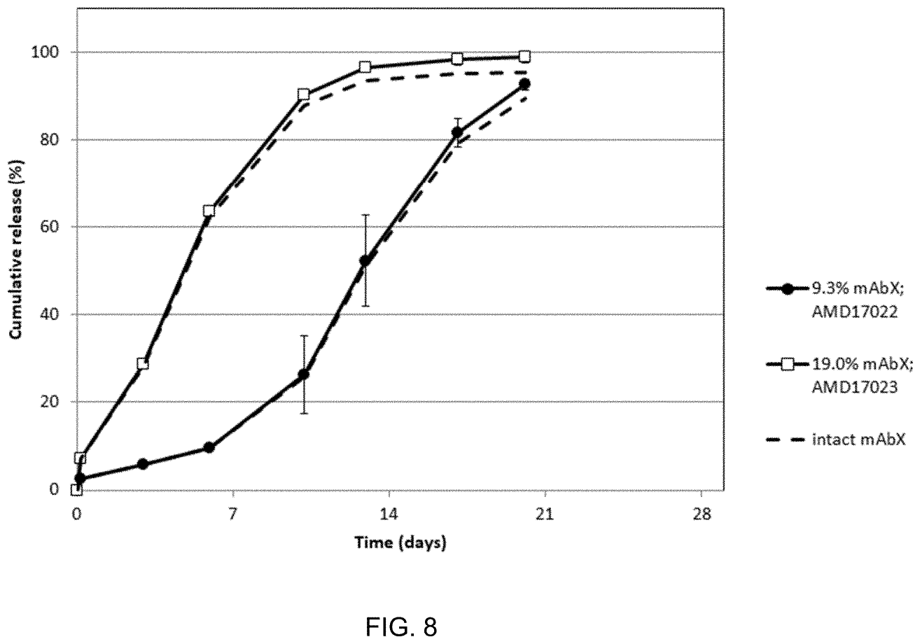

AMD17022 and AMD17023 were analysed for their in vitro release kinetics (FIG. 8). mAbX release from the mAbX MSP with 9.3 wt. % mAbX loading (AMD17022) exhibited the typical sigmoidal release kinetics but with slightly slower overall release as compared to mAbX MSP with similar mAbX loading that were prepared via the W/O/W microencapsulation process (e.g. AMD17038). mAbX MSP with 19 wt. % mAbX loading (AMD17023) showed significantly faster release with low burst release followed by linear release for almost 14 days. Both AMD17022 and AMD17023 released highly intact mAbX (integrity >96% as determined via SEC-UPLC).

### Example 5—Production of SynBiosys mAbX Microspheres with High mAbX Loading for In Vitro and In Vivo Characterisation

To allow more extensive in vitro and in vivo characterisation of the lead formulation, mAbX MSP with a target loading of 19 wt. % were prepared of a 90/10 w/w blend of 50CP10C40-LL40 and 50CP10C20-LL40 at a scale of 10 g via the W/O/O microencapsulation process using the settings used for AMD17023 (see Table 6 in Example 4). In brief, 9.0 g of 50CP30C40-LL40 (RCP-1667) and 1.0 g of 50CP10C20-LL40 (RCP-1440) were dissolved in dichloromethane to a concentration of 10 wt. % and filter sterilised through a 0.2 μm PTFE filter. The polymer solution (01) was emulsified with a 220.0 mg/ml aqueous solution of mAbX (220 mg/ml, pH 5.0) (W₁) at an O₁/W₁ ratio of 9.5 using an in-line Ultra Turrax (12 000 rpm) to yield a primary emulsion (DP). DP was directly homogenised with silicone oil using an in-line Ultra Turrax (12 000 rpm) at a silicone oil to dichloromethane ratio of 0.75 w/w. The embryonic microspheres were continuously transferred to a vessel containing 6.7 l of heptane to extract dichloromethane and silicone oil from the embryonic microspheres (final heptane to dichloromethane ratio of 13.4 (w/w)). After completion of solvent extraction the microspheres were collected by filtration, washed with heptane and finally dried overnight at 40° C. under reduced pressure using a Nutsche filter setup. Dried mAbX MSP were sieved through a 200 μm sieve to remove oversized particles and stored at −18° C. until further use.

The mAbX MSP (AMD18012) were characterised using the methods described in Example 2. mAbX-MSP were spherical and had a smooth surface without any visible pores. The microspheres had a broad particle size distribution (CV 65%) with an average particle size (D50) of 83 μm and an mAbX loading of 14.9 wt. % representing an encapsulation efficiency of 80%. Residual content of dichloromethane and heptane determined by GC-headspace (Agilent 6850 gas chromatograph equipped with a Combi-Pal headspace sampler) using DMSO to dissolve the samples (50-500 mg), octane as an internal standard and flame-ionisation detection, were <18 ppm and <357 ppm, respectively.

In vitro release kinetics of the mAbX-MSP were near-identical to AMD17023 (FIG. 8) with linear release kinetics for up to 10 days without any burst and a recovery (fraction of encapsulated mAbX released) of 86% after 2 weeks of in vitro release.

### Example 6—In Vitro Characterisation of mAbX Formulations

In this example, samples collected during in vitro release testing of mAbX MSP prepared in Example 5 were further characterised for (1) aggregation and fragmentation of mAbX by size exclusion chromatography (SEC). (2) integrity of the tertiary structure by fluorescence spectroscopy (FLS), (3) integrity of the secondary structure by circular dichroism spectroscopy (CD), (4) protein concentration by photometric determination and (5) ability to bind to its target by ELISA for quantification of functional mAbX.

### Analytical Methods

Aggregation and fragmentation of mAbX in in vitro release samples were analysed by size exclusion chromatography (SEC). Furthermore, mAbX monomer fraction was quantified. Samples were examined with a TSKgel Super SW3000 size exclusion column (TOSOH) under usage of a mobile phase (0.05 M sodium phosphate, 0.4 M sodium perchlorate, pH 6.3) and Gel Filtration Standard (Bio-Rad). Different migration times of protein aggregates, monomers and fragments through the column enables the detection of peptide bonds by UV at 214 nm of separated eluted fractions and evidence for the molecular weight. Area under the curve was used for the calculation of the percentage of eluted fractions.

Integrity of the tertiary structure of mAbX was analysed by fluorescence spectroscopy (FLS) by excitation of tryptophan at a wavelength of 280 nm. Resulting emission was recorded between 290 and 450 nm. Emission wavelength of tryptophan is shifted in case of environmental changes of the molecule resulting in information about the conformational state of the protein. The proportion of correctly folded protein in samples was determined by the calculation of the relation between the emission maxima of the sample and control samples of intact and denatured protein.

Integrity of the secondary structure of mAbX was analysed by circular dichroism (CD). The optically active peptide bonds of the intact antibody absorb left and right polarised light to a certain extent, resulting in polarised light of a specific ellipticity. The secondary structure of mAbX was analysed at a wavelength of 218 nm representing the signal of β-sheets, which are the dominating secondary structure element in antibodies. The proportion of correctly folded antibody in samples was determined by the relation between CD signals of the sample and control samples of intact and denatured mAbX in a suitable buffer system.

The protein concentration of samples was determined spectrophotometrically. Absorbance of aromatic amino acids as well as disulphide bonds in proteins were detected at a wavelength of 280 nm and used to determine the protein concentration. After entering the reciprocal value of the absorption coefficient for the specific proteins, the concentration with mg/ml output format was calculated automatically by the Eppendorf Biophotometer plus.

For ELISA analysis, a 96-well plate was coated with soluble mAbX receptor and used to capture mAbX from in vitro and in vivo samples. In this way, solely functional mAbX with the ability to bind its target was immobilised. Subsequently, an anti-human detection antibody coupled to horseradish peroxidase (HRP) was bound to captured mAbX molecules. The resulting antigen-antibody-antibody-HRP complex was quantified by HRP-driven conversion of the chromogenic substrate Tetramethylbenzidine (TMB). The colour intensity of the converted product was proportional to the amount of functional mAbX bound from the sample.

### Results

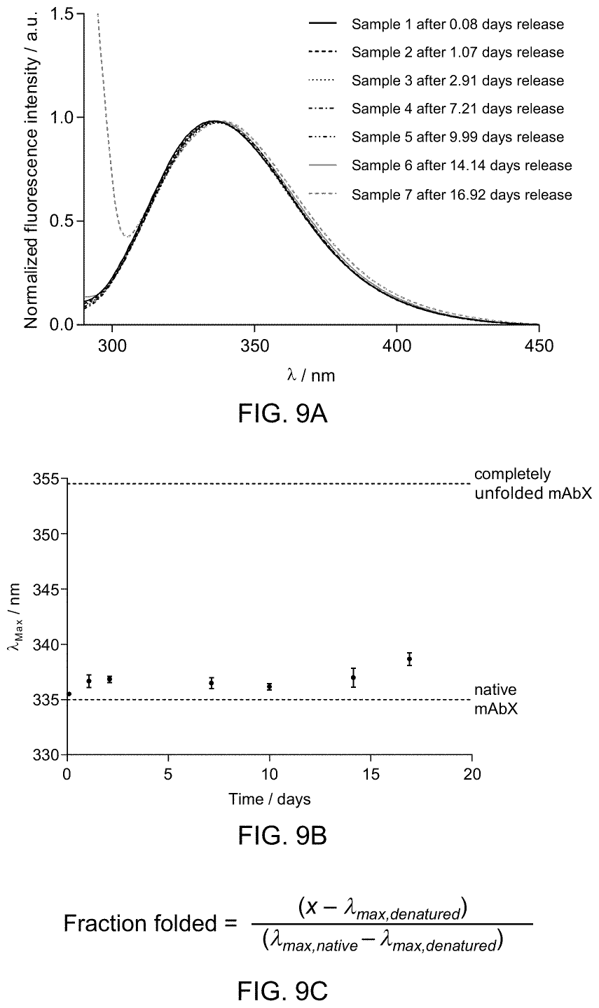

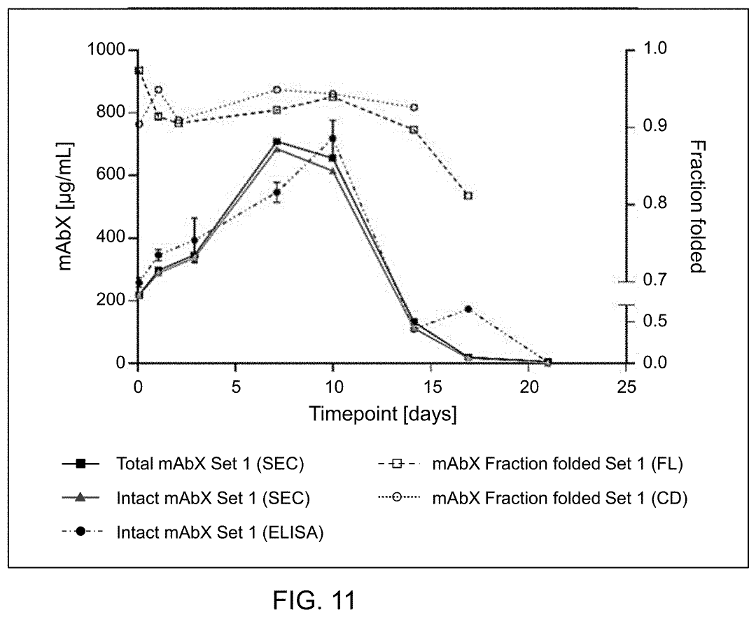

In FIG. 9 fluorescent spectroscopy (FLS) data is shown for a series of 7 mAbX samples collected from in vitro release testing of mAbX MSP (FIG. 9, A). During the time period of up to 14 days no major change within the folding characteristics of released mAbX could be demonstrated. while only at day 17 a minor red shift of the emission maximum by 2 nm was detected in comparison to native control mAbX sample (FIG. 9, B). The ratio of wavelength at the emission maximum (FIG. 9. C) was used to assess the folded vs. unfolded fractions and used to compare the FLS data with Circular Dichroism data (see also FIG. 11).

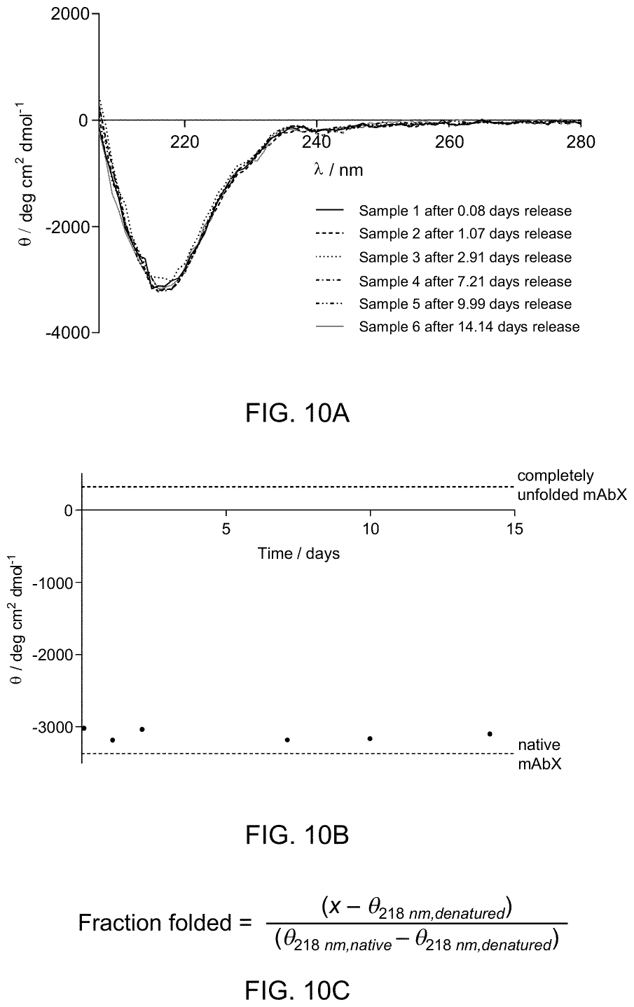

In FIG. 10 Circular Dichroism data is shown for a series of 7 mAbX samples after an in vitro release experiment (FIG. 10, A). During the time period of up to 14 days no major change within the secondary structure of mAbX occurred (FIG. 10, B). As protein concentrations of samples collected after day 14 were below the detection limit of the CD method no CD analysis could be performed for these later time points. The ratio of the signal peak at 218 nm divided by the completely unfolded mAbX signal was used to compare the CD data with FLS data (see also FIG. 11).

Finally, the data obtained from FLS and CD (fraction folded) was plotted against the total protein concentration obtained by SEC-UPLC, as well as intact monomer species of mAbX measured by SEC (FIG. 11). Controlled release of intact, correctly folded and active mAbX over a period of 14 days could be shown. After day 14 only small amounts of mAbX were released by the polymer based microsphere formulation.

In summary, the data obtained from SEC-UPLC, fluorescence spectroscopy, circular dichroism, and ELISA proof the compatibility of the [PCL-PEG-PCL]-b-[PLLA]-based multi-block copolymers and the microencapsulation process with the monoclonal antibody mAbX, a large protein of 145.8 kDA.

### Example 7—In Vivo Pharmacokinetics of mAbX MSP and Anti-Tumour Effects

mAbX MSP prepared according to Example 5 were evaluated in healthy NMRI mice for their in vivo pharmacokinetics.

Female NMRI mice were used at 4-6 weeks of age. Mice were randomly separated into 5 groups of 6 animals and received either a single injection of 1 mg mAbX control solutions by intravenous (i.v.) or subcutaneous (s.c.) injections, or a single subcutaneous injection of suspensions of mAbX MSP in aqueous reconstitution medium (water for injection, 0.6 wt. % carboxymethyl cellulose (CMC) (1, 4 or 8 mg mAbX per mouse). Blood samples were collected at indicated time points and mAbX serum concentrations were measured using a validated ELISA.

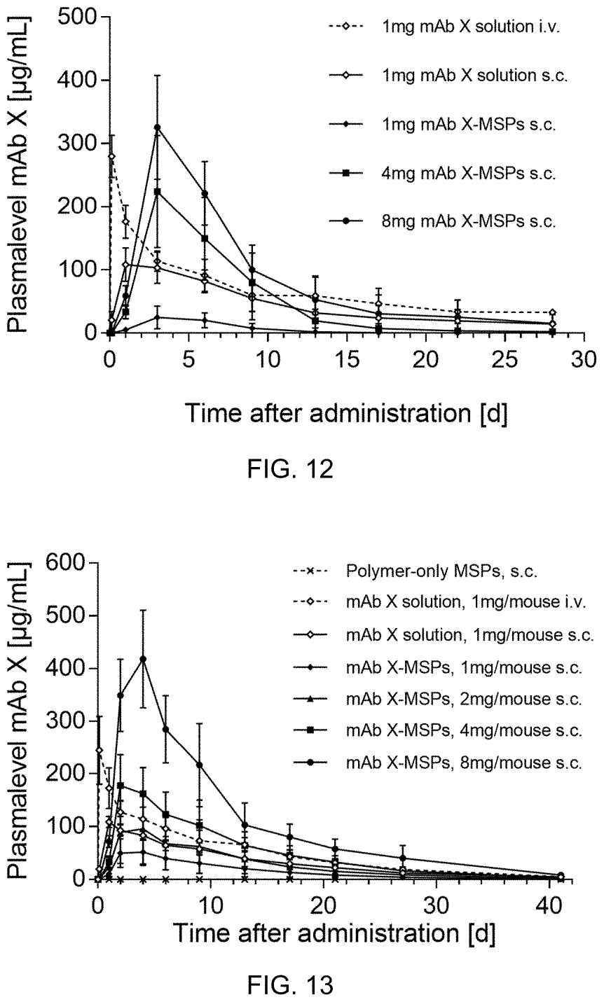

FIG. 12 shows the plasma levels of mAbX following administration. Within the i.v. control group, a plasma peak level was obtained after 3 h, while within the s.c. control group a plasma peak level was obtained after one day. Injections of the mAbX MSP formulations resulted in a shift of plasma peak level to day 3-4 with a dose dependent release of mAbX over a time period of up to 23 days within the highest dose group. The data clearly shows that mAbX is released from the microsphere formulations in vivo over an extended period of time. After in vivo release, mAbX could be detected in plasma by a binding ELISA which shows that released mAbX can still bind to its receptor indicating that the protein structure is functionally intact.

In a second pharmacokinetic study mAbX plasma levels were monitored in A431 xenograft bearing NMRI nude mice (FIG. 13) and the mAbX MSP were evaluated for their anti-tumour effect. Subcutaneous tumours were induced by inoculation of 1×10⁷ A431 cells in the flank of NMRI nude mice. When tumour volume reached 50-250 mm³, mice were randomly assigned to each treatment group (n=7). The mice received a single injection of either mAbX-solution (1 mg/mouse) or mAbX-loaded microspheres (0-8 mg mAbX/mouse). Blood samples were collected at indicated time points and mAbX serum concentrations were measured using a validated ELISA. Tumour volume was determined by Calliper measurement twice weekly.

Control injections of 1 mg mAbX i.v. resulted in a plasma peak after 3 h, while after a control injection of 1 mg mAbX solution s.c., a plasma peak after 1 day could be observed. mAbX MSP formulations were injected subcutaneously with a final mAbX dose of 1, 2, 4 or 8 mg, respectively. Plasma peaks shifted to 2-4 days while a sustained release effect was observed up to 27 days for the highest dose. This second pharmacokinetic study also confirmed that mAbX is successfully released from microspheres formulations in vivo. mAbX could be detected in plasma by a functional ELISA, which proofs that the protein structure is still functionally intact.

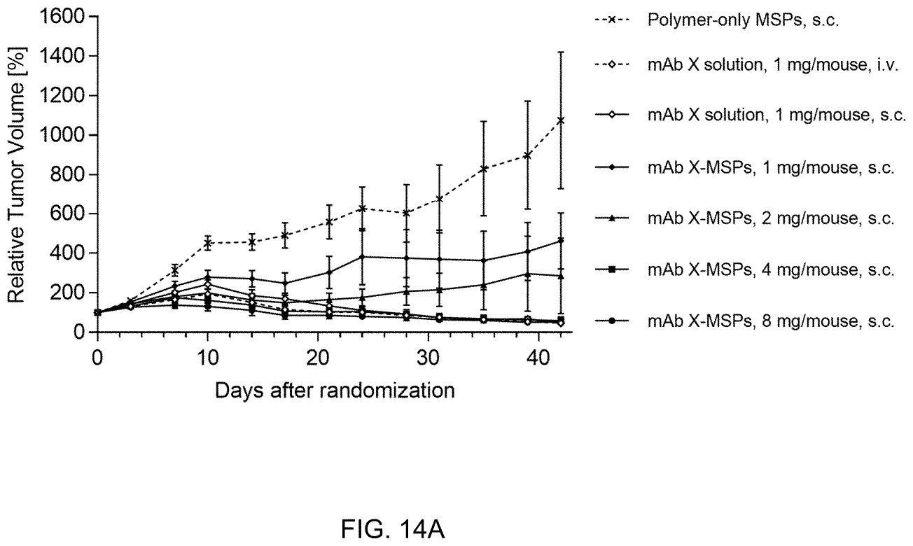

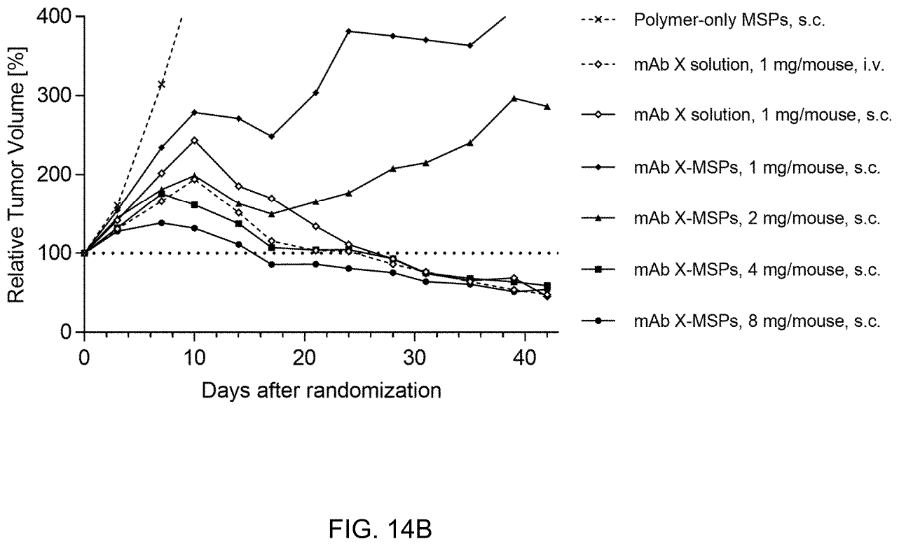

In FIG. 14 tumour growth in A431 tumour bearing NMRI nude mice was monitored. Subcutaneous tumours were induced by inoculation of 1×10⁷ A431 cells in the flank of NMRI nude mice. When tumour volume reached 50-250 mm³, mice were randomly assigned to each treatment group (n=7). Tumour volume was determined by Calliper measurement twice weekly. After injection of a polymer only group (without any mAbX), tumours were constantly growing over a time period of 42 study days. Significant anti-tumour effects (p≤0.002) could be shown with a mAbX i.v. injection as well as an 8 mg mAbX MSP formulation after 7 days, and with mAbX s.c. solution as well as a 1, 2 or 4 mg mAbX MSP formulation after 10 days. With this data set we could prove, that mAbX is successfully released from the microsphere formulations in vivo and that after release the protein is still structurally intact and biologically active.

### Example 8—Production of Sustained Release Microspheres of mAb02

In another study, a second monoclonal antibody with a molecular weight of 144.9 kDa (mAb02) was formulated into SynBiosys microspheres at a target loading of 19 wt. % by means of the W/O/O process using the same polymer compositions as used for the production of the mAbX-MSP formulations in Example 4.

In brief, a total of 1 g of polymer (representing different blend ratios of 50CP30C40-LL40 and 50CP10C20-LL40) was dissolved in 9 g of dichloromethane and emulsified (Ultraturrax, 21 600 rpm, 40 s) with a 221.4 mg/ml mAb02 solution at a W1/O ratio of 9.5. The primary emulsion was subsequently homogenised (Ultraturrax, 21 600 rpm, 30 s) with 5.3 g silicon oil (350 CST), after which the embryonic microspheres were transferred into a vessel containing 250 ml n-heptane to extract dichloromethane and silicone oil for 1 hour. The mAb02 MSP were collected on a 5 μm filter, washed twice with 250 ml n-heptane and finally dried overnight at 40° C. under reduced pressure.

Microspheres were generated according to this method for a variety of polymer blend ratios, including those listed in Table 7.

TABLE 7
Characteristics of mAb02-loaded MSP prepared via W/O/O
microe neap sula tion route.

| | Weight fraction 50CP30C40-LL40 | | | | mAb02 | Encaps. |
|---|---|---|---|---|---|---|
| | and 50CP10C20-LL40 | | D50 | CV | content | efficiency |
| Batch nr. | 50CP30C40-LL40 | 50CP10C20-LL40 | (μm) | (%) | (wt. %) | (%) |
| SR19-002.A | 100% | 0% | 68.5 | 63% | 18.0% | 93.7% |
| SR19-002.B | 90% | 10% | 62.0 | 60% | 17.5% | 91.0% |
| SR19-002.C | 80% | 20% | 88.9 | 71% | 18.4% | 95.8% |
| SR19-002 D | 70% | 30% | 67.1 | 77% | 17.7% | 92.1 % |
| SR19-002.E | 50% | 50% | 57.7 | 59% | 18.1% | 94.5 % |
| SR19-002.F | 25% | 75% | 48.0 | 63% | 17.8% | 92.5 % |

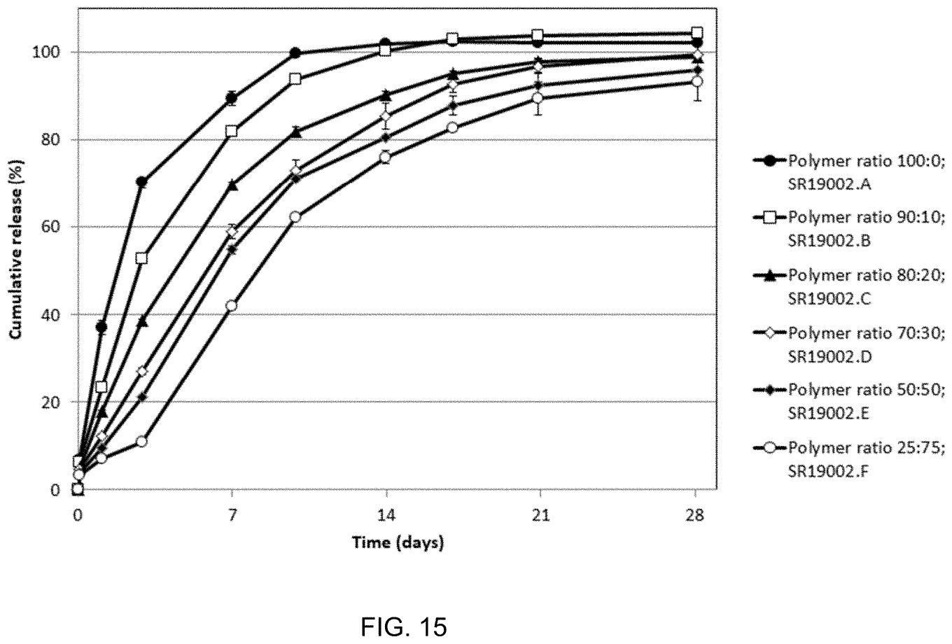

The resulting mAb02 MSP were characterized for particle size distribution, mAb02 content and mAb02 in vitro release kinetics using the same methods as used for mAbX and described in the previous examples. The average particle size varied between 57 and 89 μm. The results show that the above described coacervation process allows encapsulation of high mAb02 contents (17.7-18.4 wt. %) in microspheres maintaining a high encapsulation efficiency (>91%). mAb02 in vitro release kinetics of the various microsphere formulation are displayed in FIG. 15. All formulations exhibit a low burst release of 5 5% during the first 2 hours. Total mAb02 release duration is increased from approximately 1 week to approximately 4 weeks by decreasing the weight fraction of the 50CP30C40-LL40 relative to 50CP10C20-LL40.

For mAb02 MSP composed of a blend of 90 wt. % 50CP30C40-LL40 and 10 wt. % 50CP10C20-LL40 (SR19-002.B) the integrity of the released mAb02 was determined via SEC-UPLC by calculating the ratio of the peak areas of the intact mAb02 and the mAb02 related soluble aggregates. No additional peaks were observed in the chromatograms. Table 8 shows the total released mAb02 and the purity of released mAb02 as determined by SEC-HPLC.

TABLE 8
Purity of mAb02 released from mAb02 MSP prepared
via W/O/O microencapsulation route (SR19-002.B).

| Time | Cumulative | Purity released |
|---|---|---|
| (days) | release (%) | mAb02 (%) |
| 0.1 | 6.4 | 89% |
| 1 | 23.4 | 87% |
| 3 | 52.9 | 89% |
| 7 | 81.9 | 88% |
| 10 | 93.7 | 86% |
| 14 | 100.4 | 80% |

Typically the purity of released mAb02 was 86-89%. The lower purity obtained at 14 days (80%) is attributed to the low concentration of mAb02 at that time point. Overall, close to around 90% of the microencapsulated mAb02 is released in its intact form from mAb02 MSP (as determined by SEC-UPLC).

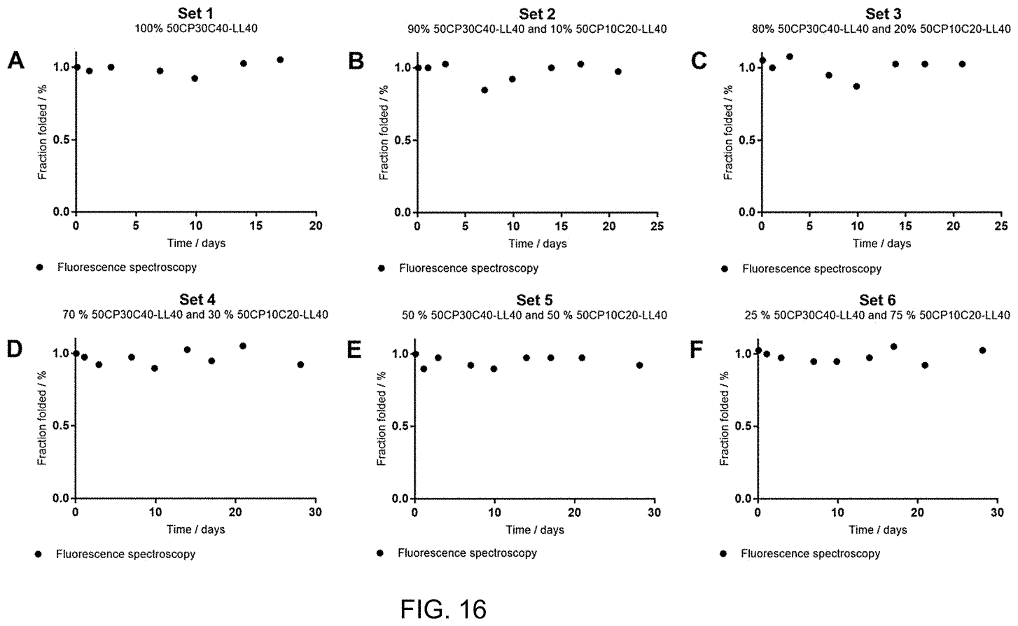

In FIG. 16 fluorescence spectroscopy (FLS) data is shown after in vitro release from mAb02 microsphere formulations utilising different polymer blends (FIGS. 16 A to F). Within these different polymer blend ratios, the hydrophilicity of the polymer blends is decreasing from A to F. The FLS data shows that the intrinsic fluorescent tryptophan spectra of mAb02 is unaffected by different polymer compositions.

### Example 9—In Vitro Erosion Kinetics of 50CP10C20-LL40

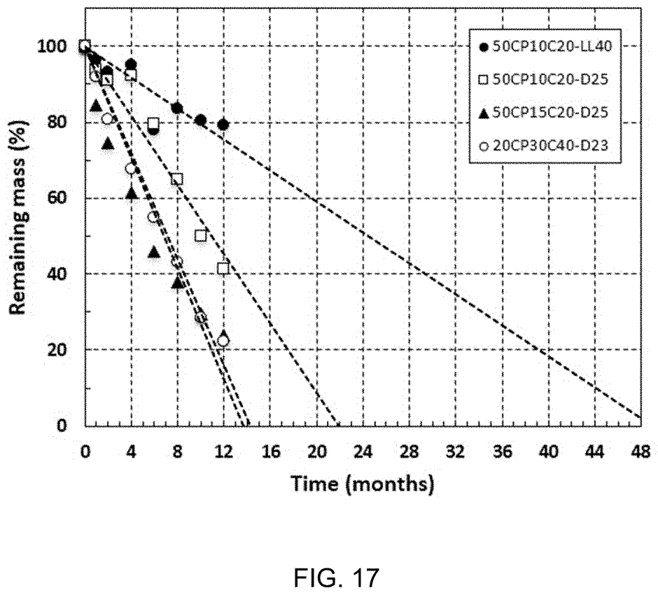

Unfortunately, polymer-only microspheres composed of the poly(L-lactide)-based 50CP10C20-LL40 multi-block copolymer, prepared as described in Example 2 using an oil-in-water (O/W)-based solvent extraction/evaporation emulsification process, were found to degrade very slowly (pH 7.4, 37° C.). Based on extrapolation of the experimentally determined remaining mass of the microspheres up to 12 months, the in vitro erosion time for 50CP10C20-LL40 microspheres is projected to be˜4 years (FIG. 17). The slow erosion of poly(L-lactide)-based multi-block copolymers was confirmed for other poly(L-lactide) such as 30CP30C40-LL40 and attributed to slow hydrolysis of the crystalline poly(L-lactide) block.

Poly(p-dioxanone) (PDO)-based multi-block copolymers, prepared by chain extending PDO pre-polymer blocks, with a Mₙ of around 2500 g/mol in different weight ratios with various [PCL-PEG-PCL] pre-polymers, were found to degrade significantly faster as compared to the poly(L-lactide) based multi-block copolymers (FIG. 17).

Polymer-only microspheres composed of 50CP10C20-D25, were found to degrade approximately twice as fast as 50CP10C20-LL40. By using higher molecular weight PEG, such as PEG1500 or PEG3000, the erosion rate increased even further, as is shown for 50CP15C20-D25, a [PCL-PEG1500-PCL]-b-[PDO] multi-block copolymer with a block ratio of 50/50 and 20CP30C40-D23, a [PCL-PEG3000-PCL]-b-[PDO] multi-block copolymer with a block ratio of 20/80 (FIG. 17).

### Example 10—Sustained Release Microspheres of mAb02 Prepared of [PCL-PEG-PCL]-b-[PDO] Multi-Block Copolymers

This example describes the production of mAb02 MSP composed of [PCL-PEG-PCL]-b-[PDO] multi-block copolymers with improved polymer degradation kinetics.

mAb02 MSP with a target mAb02 loading of 20% were produced of blends of 50CP30C40-D25 and 50CP10C20-D25 (synthesised as described in Example 1) and a mAb02 solution with a concentration of 221.4 mg/ml at a scale of 1 g according to the same procedures as described in Example 8. The mAb02 MSP were characterised for particle size distribution. mAb02 content, mAb02 in vitro release kinetics and integrity of released mAb02 using the same methods described in Example 8.

The resulting mAb02 MSP had significantly larger particle size (˜120-330 μm) than mAbX and mAb02 microspheres generated from blends of 50CP30C40-LL40 and 50CP10C20-LL40 (Table 9). Microscopic examination by SEM showed extensive agglomeration which explained the relatively large particle size as measured by laser diffraction.

TABLE 9
Characteristics of mAb02-loaded MSP prepared of
[PCL-PEG-PCL]-b-[PDO] polymers via W/O/O microencapsulation route.

| | Weight fraction 50CP30C40-D25 and | | | | mAb02 | Encaps. |
|---|---|---|---|---|---|---|
| | 50CP10C20-D25 | | D50 | cv | content | efficiency |
| Batch nr. | 50CP30C40-D25 | 50CP10C20-D25 | (μm) | (%) | (wt. %) | (%) |
| ND1901-7A | 100% | 0% | 125.6 | 53% | 13.2% | 66% |
| NK1901-69 | 90% | 10% | 331.9 | 92% | 17.3% | 87% |
| ND1901-7B | 80% | 20% | 156.4 | 66% | 14.4% | 72% |
| ND1901-70 | 70% | 30% | 135 6 | 74% | 16.2% | 81% |
| ND1901-7D | 50% | 50% | 133.0 | 51% | 15.7% | 78% |
| ND1901-7E | 25% | 75% | 123.2 | 56% | 14.9% | 74% |
| ND1901-7F | 0% | 100% | 191.1 | 77% | 15.9% | 79% |

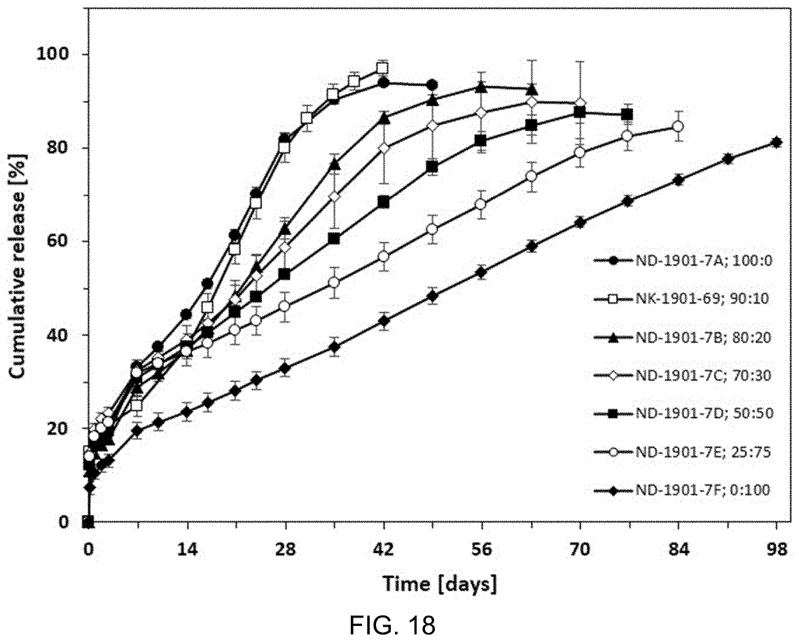

Total protein content as determined using the BCA-based content analysis method showed mAb02 contents varying from 13.2 to 17.3 wt. %, representing encapsulation efficiencies varying from 66% to 87%, which was significantly lower as compared to the typical encapsulation efficiencies of >90% obtained for mAb02 MSP prepared of blends of 50CP30C40-LL40 and 50CP10C20-LL40. mAb02 in. vitro release kinetics of the various microsphere formulations are displayed in FIG. 18.

All formulations exhibited a moderate burst release of 10-15% which was followed by sustained release with almost perfectly linear release kinetics thereafter. mAb02 MSP prepared of 100% 50CP30C40-D25 release mAb02 over 6 weeks. By (partly) replacing 50CP30C40-D25 by 50CP10C20-D25 (blend ratios 90:10 to 0:100). the release rate of mAb02 could be effectively decreased yielding mAb02 MSP formulations with durations of release varying from 2 months (blend ratios 80:20 and 70:30) to 3-4 months (100% 50CP10C20-D25). The integrity of released mAb02 was determined via SEC-UPLC by calculating the ratio of the peak areas of the intact mAb02 and the mAb02 related soluble aggregates. No additional peaks were observed in the chromatograms. Table 10 shows the total released mAb02 and the purity of released mAb02 as determined by SEC-HPLC for mAb02 MSP composed of a 90:10 blend of 50CP30C40-D25 and 50CP10C20-D25. After 6 weeks, approximately 85% of the total released mAb02 is still released in its intact form confirming compatibility of mAb02 with the used polymers and manufacturing procedures.

TABLE 10
Purity of mAb02 released from mAb02 MSP prepared
of [PCL-PEG-PCL]-b-[PDO] based SynBiosys
polymers via W/O/O microencapsulation route (NK1901-69).

| Time | Cumulative | Purity released |
|---|---|---|
| (days) | release (%) | mAb02 (%) |
| 0.1 | 15.1 | 89.5 |
| 1 | 18.6 | 90.0 |
| 3 | 20.8 | 87.1 |
| 7 | 24.8 | 81.4 |
| 14 | 37.7 | 83.5 |
| 21 | 58.3 | 91.5 |
| 28 | 80.1 | 90.8 |
| 35 | 91.5 | 72.5 |
| 38 | 94.3 | 51.1 |
| 42 | 97.0 | 28.7 |

Characterisation of in vitro released mAb02 by FLS according to the method described in Example 6 showed that the intrinsic fluorescent tryptophan spectra of mAb02 were unaffected after release from microsphere formulations composed of different 50CP30C40-D25/50CP10C20-D25 polymer blends.

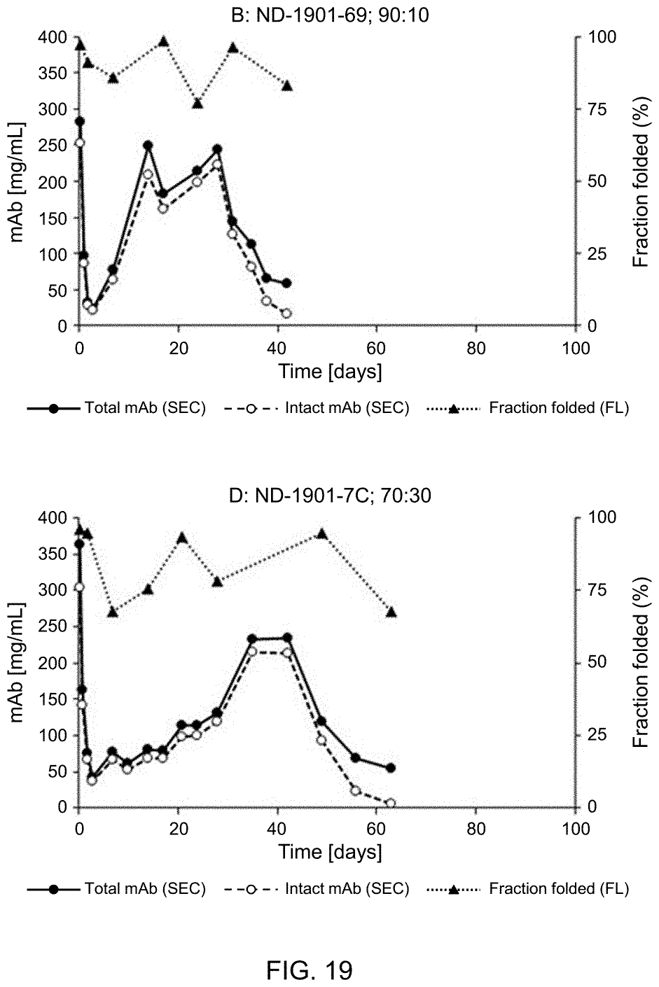

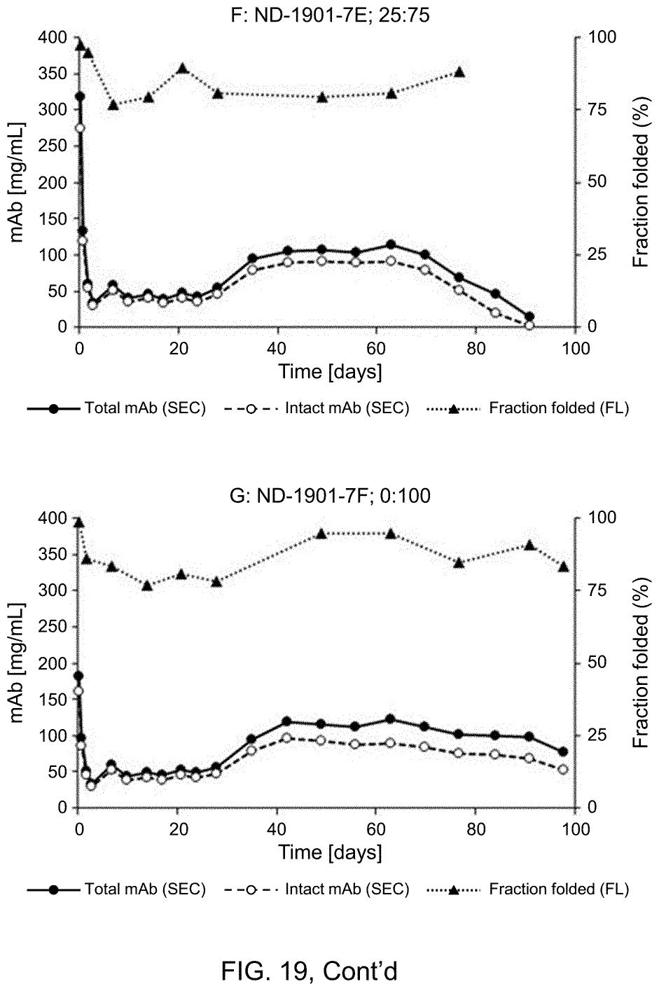

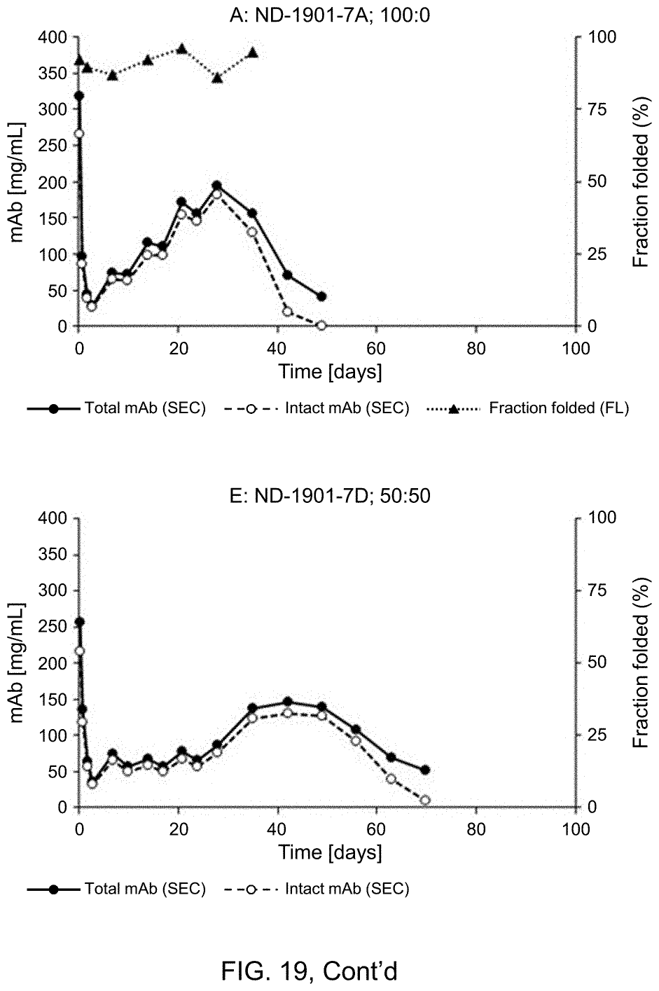

FIG. 19 shows the concentration of total and intact mAb02 (as measured by SEC-UPLC) as well as the fraction correctly folded mAb02 (as determined by fluorescence spectroscopy (FLS) according to the method shown in FIG. 9 C), in in vitro release samples collected at different time points during in vitro release testing of mAb02-loaded microsphere formulations composed of different 50CP30C40-D25/50CP10C20-D25 polymer blends (FIG. 19 A to G). Within these different polymer blend ratios, the hydrophilicity of the polymer blends is decreasing from A to G.

The fraction intact mAb02 as determined by SEC-UPLC was typically 80-90%. The fraction correctly folded mAb02 as determined by FLS varied from 65 to 100%, irrespective of the duration of release and was not affected by the composition of the polymer blend.

In summary, the data obtained from SEC-UPLC and fluorescence spectroscopy show that release of intact and correctly folded mAb02 for periods of up to 3 months is feasible and proof the compatibility of the faster degrading [PCL-PEG-PCL]-b-[PDO]-based multi-block copolymers and the microencapsulation process with the monoclonal antibody mAb02, a large protein of 144.9 kDA

1. A dosage form for extended release of an antibody or an antigen binding fragment thereof, comprising
(a) an antibody or an antigen binding fragment thereof;
(b) a biodegradable multi-block copolymer matrix,
wherein the antibody or the antigen binding fragment thereof is present in the multi-block copolymer matrix,
wherein the biodegradable multi-block copolymer comprises one or more biodegradable, phase separated, thermoplastic multi-block copolymers comprising at least one amorphous hydrolysable pre-polymer (A) segment and at least one semi-crystalline hydrolysable pre-polymer (B) segment, wherein
said multi-block copolymer under physiological conditions has a T_{g} of about 37° C. or less and a Tₘ of about 50° C. to about 110° C.;
the segments are linked by a multifunctional chain extender;
the segments are randomly distributed over the polymer chain; and
the pre-polymer (B) segment comprises a X—Y—X triblock copolymer wherein Y is a polymerisation initiator, and X is a poly(p-dioxanone) segment with a block length expressed in p-dioxanone monomer units of about 7 or more.

2. The dosage form of claim 1, wherein the multi-block copolymer matrix releases less than about 3% to about 40% of the antibody or the antigen binding fragment thereof based on total weight of antibody or antigen binding fragment thereof present in the multi-block copolymer matrix within about 24 hours.

3. The dosage form of claim 1, wherein the antibody or the antigen binding fragment thereof comprises one or more selected from the group consisting of monoclonal antibodies, bispecific antibodies, tri-specific antibodies, antibody drug conjugates, antigen-binding fragments include Fab, F(ab′), F(ab′)₂, single-chain antibodies (scFv) and bivalent single-chain antibodies.

4. The dosage form of claim 1, wherein the antigen binding fragment comprises at least two paired domains.

5. The dosage form of claim 1, wherein the antibody or the antigen binding fragment thereof has a molecular weight of about 70 kDa or more.

6. The dosage form for extended release of a protein of about 70 kDa or more, comprising
(a) a protein of about 70 kDa or more;
(b) a biodegradable multi-block copolymer matrix,
wherein the protein is present in the multi-block copolymer matrix,
wherein the biodegradable multi-block copolymer comprises one or more biodegradable, phase separated, thermoplastic multi-block copolymers comprising at least one amorphous hydrolysable pre-polymer (A) segment and at least one semi-crystalline hydrolysable pre-polymer (B) segment, wherein
said multi-block copolymer under physiological conditions has a T_{g} of about 37° C. or less and a Tₘ of about 50° C. to about 110° C.;
the segments are linked by a multifunctional chain extender;
the segments are randomly distributed over the polymer chain; and
the pre-polymer (B) segment comprises a X—Y—X triblock copolymer wherein Y is a polymerisation initiator, and X is a poly(p-dioxanone) segment with a block length expressed in p-dioxanone monomer units of about 7 or more.

7. The dosage form according to claim 6, wherein the protein comprises one or more selected from the group consisting of Fc fusion proteins, antibody drug conjugates (ADCs), full length immunoglobulins, coagulation factors, growth factors, hormones, cytokines, and enzymes.

8. The dosage form of claim 1, wherein the pre-polymer (B) segment comprises about 70% or more of poly(p-dioxanone) by total weight of said pre-polymer (B) segment.

9. The dosage form of claim 1, wherein the block length of the poly(p-dioxanone) segment X expressed in terms of p-dioxanone monomer units is about 7 to about 35.

10. The dosage form of claim 1, wherein the polymerisation initiator Y is a polymerisation initiator selected from the group consisting of ethylene glycol, 1,2-propanediol, 1,3-propanediol, 1,4-butanediol, 1,3-butanediol, 2,3-butanediol, diethylene glycol, dipropylene glycol, triethylene glycol, poly(ethylene glycol), 1,5-pentanediol, 1,6-hexanediol, neopentyl glycol, hydrogenated bisphenol A, and glycerol.

11. The dosage form of claim 1, wherein the pre-polymer (B) segment has a number average molecular weight Mₙ of about 1300 g/mol or more to about 7200 g/mol.

12. The dosage form of claim 1, wherein the pre-polymer (B) segment has weight average molecular weight M_{w} of about 1800 g/mol to about 10 080 g/mol.

13. The dosage form of claim 1, wherein the content of the pre-polymer (B) segment in the copolymer is about 5% to about 95% by total weight of the multi-block copolymer.

14. The dosage form of claim 1, wherein the pre-polymer (A) segment comprises reaction products of one or more selected from the group consisting of glycolide, lactide (d and/or 1), ε-caprolactone, δ-valerolactone, trimethylene carbonate, tetramethylene carbonate, 1,5-dioxepane-2-one, 1,4-dioxane-2-one (p-dioxanone), and cyclic anhydrides.

15. The dosage form of claim 1, wherein the pre-polymer (A) comprises reaction products of glycolide, lactide (d and/or 1), and/or ε-caprolactone.

16. The dosage form of claim 1, wherein about 30% or more by total weight of pre-polymer (A) is derived from a water-soluble polymer.

17. The dosage form of claim 1, wherein the water-soluble polymer comprises one or more selected from the group consisting of poly(ethylene glycol) (PEG), poly(tetramethylene oxide) (PTMO), poly(propylene glycol) (PPG), poly(vinylalcohol) (PVA), poly(vinylpyrrolidone) (PVP), poly(vinylcaprolactam), poly(hydroxyethylmethacrylate) (poly-(HEMA)), poly(phosphazenes), poly(orthoesters), poly(orthoesteramides), and copolymers of any of these polymers.

18. (canceled)

19. The dosage form of claim 1, wherein the water-soluble polymer comprises, or is, poly(ethylene glycol).

20. The dosage form of claim 1, wherein the pre-polymer (A) segment comprises poly(ε-caprolactone)-co-PEG-co-poly(ε-caprolactone).

21. The dosage form of claim 1, wherein the pre-polymer (A) segment has a number average molecular weight Mₙ of about 500 g/mol to about 10 000 g/mol.

22. The dosage form of claim 1, wherein the content of pre-polymer (A) in the multi-block copolymer is from about 5% to about 95% based on total weight of the multi-block copolymer.

23. (canceled)

24. The dosage form of claim 1, wherein the multifunctional chain extender is a diisocyanate.

25. The dosage form of claim 1, wherein the dosage form is in the form of one or more selected from the group consisting of microspheres, microparticles, nanospheres, nanoparticles, a rod, an implant, a film, a sheet, a tube, a membrane, a mesh, fibres, a plug, a coating, and a gel.

26. (canceled)

27. The dosage form of claim 1, wherein the dosage form is in the form of microspheres having an average diameter of from about 1 μm to about 200 μm.

28. A method of administering the dosage form of claim 1, wherein the multi-block copolymer matrix releases less than about 20% of protein or antibody or antigen binding fragment thereof based on total weight of protein or antibody or antigen binding fragment thereof present in the multi-block copolymer matrix within about 24 hours.

29. The method of claim 28, wherein the administration is via intradermal, transdermal, intramuscular, subcutaneous, intravitreal, intraarticular, or intratumoural injection.

30. A method of treating a subject in need of a protein or an antibody or antigen binding fragment thereof, comprising administering the dosage form of claim 1.

31.-32. (canceled)
